(19) United States
(12) Patent Application Publication
(10) Pub. No.: US 2010/0266672 A1
(43) Pub. Date: Oct. 21, 2010

(54) VACCINES

(76) Inventors: Nathalie Marie-Josephe Garcon, Rixensart (BE); Emmanuel Jules Hanon, Rixensart (BE)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(21) Appl. No.: 12/095,537

(22) PCT Filed: Nov. 28, 2006
(86) PCT No.: PCT/EP06/01469

§ 371 Date: May 30, 2008

(30) Foreign Application Priority Data
Nov. 30, 2005 (GB) 0524409.0

(51) Int. Cl. A61K 39/112; A61K 39/39; A61P 37/04; A61K 9/127

(52) U.S. Cl. 424/450; 424/197.11; 424/194.1; 424/236.1

(57) ABSTRACT

The present invention provides a vaccine composition comprising the B subunit of Shiga toxin or an immunologically functional equivalent thereof which is able to bind the Gb3 receptor, complexed with at least one first antigen, and further comprising at least one second antigen (which may be the same or different as the first antigen) and an adjuvant.

The present invention provides improved vaccine compositions, methods for making them and their use in medicine. In particular the present invention provides adjuvanted vaccine compositions which comprise the B sub unit of Shiga Toxin or an immunologically functional equivalent thereof complexed with a first antigen and a second antigen which may be the same or different to the first antigen, said composition being formulated with an adjuvant.

U.S. Pat. No. 6,613,882 discloses a chimeric polypeptide of the formula: B-X wherein B represents the B fragment of Shiga toxin or a functional equivalent thereof, and X represents one or more polypeptides of therapeutic significance, wherein said polypeptides are compatible with retrograde transport mediated by B to ensure processing or correct addressing of X.

WO02/060937 is an application which discloses a universal polypeptidic carrier for targeting directly or indirectly to Gb3 receptor and having the formula STxB-Z(n)-Cys; wherein StxB is the shiga Toxin B subunit Z is an amino acid linker with no sulfhydryl groups n is 0, 1, 2, or polypeptide and Cys is Cysteine.

The development of vaccines which require a predominant induction of a cellular response remains a challenge. Because CD8+ T cells, the main effector cells of the cellular immune response, recognise antigens that are synthesized in pathogen-infected cells, successful vaccination requires the synthesis of immunogenic antigens in cells of the vaccinee. This can be achieved with live-attenuated vaccines however these also present significant limitations. First, there is a risk of infection, either when vaccinees are immunosuppressed, or when the pathogen itself can induce immunosuppression (e.g. Human Immunodeficiency Virus). Second, some pathogens are difficult or impossible to grow in cell culture (e.g. Hepatitis C Virus). Other existing vaccines such as inactivated whole-cell vaccines or alum adjuvanted, recombinant protein subunit vaccines are notably poor inducers of CD8 responses.

For these reasons, alternative approaches are being developed: live vectored vaccines, plasmid DNA vaccines, synthetic peptides or specific adjuvants. Live vectored vaccines are good at inducing a strong cellular response but pre-existing (e.g. adenovirus) or vaccine-induced immunity against the vector may jeopardize the efficiency of additional vaccine dose (Casimiro et al, JOURNAL OF VIROLOGY, June 2003, p. 6305-6313). Plasmid DNA vaccines also can induce a cellular response (Casimiro et al, JOURNAL OF VIROLOGY, June 2003, p. 6305-6313) but it remains weak in humans (Mc Conkey et al, Nature Medicine 9, 729-735, 2003) and the antibody response is very poor. In addition, synthetic peptides are currently being evaluated in clinical trials (Khong et al, J Immunother 2004; 27:472-477), but the efficacy of such vaccines encoding a limited number of T cell epitopes may be hampered by the appearance of vaccine escape mutants or by the necessity of first selecting for HLA-matched patients.

Alternative approaches based on antigen delivery using non-live vectors such as bacterial toxins have also been described. The Shiga B vectorisation system (STxB) is based on the non toxic B subunit of the Shiga toxin derived from Shigella dysenteriae. This molecule has a number of characteristics that seem to predispose it as a vector for antigen presentation: absence of toxicity, low immunogenicity, targeting through CD77 receptor and ability to introduce cargo antigen into the MHC class 1-restricted antigen-presentation pathway (Haicheur et al (2003) Int. Immunol 15 pp 1161-1171). In particular, the physical linkage of antigens to the B subunit of the Shiga toxin has been shown to induce detectable CD8 responses in mouse models (Haicheur et al, 2000 Journal of Immunology 165 pp 3301-3308; Haicheur et al, 2003 Int. Immunol 15 pp 1161-1171). However, this response required three injections of high amounts of antigen (up to 80 μg, Haicheur et al, 2003 Int. Immunol 15 pp 1161-1171), and could not be improved by mixing with Freund's Incomplete adjuvant when administered intra peritoneally. (Haicheur et al, 2000 Journal of Immunology 165 pp 3301-3308.)

In addition, there would be advantages to a vaccine composition that could activate, as discussed above, CD8 responses whilst at the same time activating CD4 resonses or generating a specific antibody response.

The present inventors have found that the inclusion of adjuvants in compositions comprising the B subunit of Shiga toxin or an immunologically functional equivalent thereof complexed with an antigen and further comprising at least one second antigen can have a beneficial effect on the resulting immune response. The present inventors have found that the inclusion of adjuvant enables in particular a beneficial increase in the immune response to the complexed antigen. The present inventors have also found that the inclusion of the same antigen in both free and complexed form enables the activation of both cellular and humoral immunity to the antigen. The present inventors have further found that the inclusion of one antigen in complexed form and one antigen in free form enables the activation of cellular and humoral immunity to both antigens thereby providing a complete immune response. Therefore the present invention provides a vaccine composition comprising at least one B subunit of Shiga toxin or an immunologically functional equivalent thereof which is able to bind the Gb3 receptor, complexed with a first antigen, and further comprising one or more second antigens which may be the same or different to the first antigen, and further comprising an adjuvant.

Particular adjuvants are those selected from the group of metal Salts, oil in water emulsions, Toll like receptors ligand, (in particular Toll like receptor 2 ligand, Toll like receptor 3 ligand, Toll like receptor 4 ligand, Toll like receptor 7 ligand, Toll like receptor 8 ligand and Toll like receptor 9 ligand), saponins or combinations thereof. In one embodiment the adjuvant does not include a metal salt as sole adjuvant. In one embodiment the adjuvant does not include a metal salt. In contrast to the situation demonstrated in the prior art the present inventors have shown the ability of incomplete Freund's adjuvant to augment the effect of Shiga toxin (or an immunologically functional equivalent) and antigen when such a composition is not administered intra muscularly. In addition this improvement of the CD8 response is readily observed after a single injection and when using lower doses of antigen.

The B subunit of Shiga toxin and immunologically functional equivalents thereof are herein termed proteins of the invention. Immunologically functional equivalents of the B subunit of Shiga toxin are defined as a protein such as, but not limited to, a toxin, a toxin subunit or a functional fragment thereof which is able to bind the Gb3 receptor. Such binding capability may be determined by following the assay protocol set out in example 1.2. Gb3 binding is believed to induce the appropriate transport of the antigen of interest and thereby to promote its presentation by MHC class 1. In one embodiment, such proteins have at least 50% amino acid sequence identity, preferably 60%, 70%, 80% 90% or 95% identity for example 96%, 97%, 98% or 99% identity at the amino acid level to the mature form of the B subunit of Shiga Toxin.

Such immunologically functional equivalents include the B subunit of toxins isolated from a variety of Shigella species, in particular Shigella dysenteriae. Additionally, immunologically functional equivalents of the B subunit of Shiga toxin include homologous toxins which are able to bind the Gb3 receptor from other Bacteria, which toxins preferably have at least 50% amino acid sequence identity to the B subunit of Shiga toxin. For example, the B subunit of verotoxin-1 (VT1) from E Coli is identical to the B subunit of Shiga toxin. VT1 and VT2 from E coli are known to bind the Gb3 receptor in-vitro and may be used in the context of the present invention, as well as other Shiga-like toxins produced by other bacteria. In the context of the invention, the word toxin is intended to mean toxins that have been detoxified such that they are no longer toxic to humans, or a toxin subunit or fragment thereof that are substantially devoid of toxic activity in humans.

The compositions of the invention are capable of improving a CD8 specific immune response to the antigen complexed to a protein of the invention. Improvement is measured by looking at the response to a composition of the invention comprising a first antigen complexed to a protein of the invention and a second antigen and further comprising an adjuvant when compared to the response to a composition comprising a first antigen complexed to a protein of the invention and a second antigen with no adjuvant, or the response to a formulation comprising a first and second antigen with adjuvant. Improvement may be defined as an increase in the level of the immune response, the generation of an equivalent immune response with a lower dose of antigen, an increase in the quality of the immune response, an increase in the persistency of the immune response, or any combination of the above. Such an improvement may be seen following a first immunization, and/or may be seen following subsequent immunizations.

In one embodiment of the invention low doses of antigen (as low as 8 ng antigen for a mouse), may be used to raise such an immune response. In this embodiment the adjuvanted, antigen complexed to a protein of the invention can induce a primary CD 8 response (as measured by tetramer staining, intracellular cytokine staining and in vivo cytotoxic activity) which is persistent as compared to adjuvanted antigen which is not complexed to a protein of the invention, or an antigen complexed to a protein of the invention but without adjuvant, which are unable to raise such a persistent response.

The CD8 immune response wanes over time: after the peak, there is a contraction phase where most effector cells die, while memory cells survive. The establishment of this responsive memory T cell population is appreciated by both the long-term detection of antigen-specific cells and their ability to be boosted.

The adjuvant is preferably selected from the group: a saponin, lipid A or a derivative thereof, an immunostimulatory oligonucleotide, an alkyl glucosaminide phosphate, or combinations thereof. A further preferred adjuvant is a metal salt in combination with another adjuvant. It is preferred that the adjuvant is a Toll like receptor ligand in particular an ligand of a Toll like receptor 2, 3, 4, 7, 8 or 9, or a saponin, in particular Qs21. It is further preferred that the adjuvant system comprises two or more adjuvants from the above list. In particular the combinations preferably contain a saponin (in particular Qs21) adjuvant and/or a Toll like receptor 9 ligand such as a immunostimulatory oligonucleotide containing CpG or other immunostimulatory motifs such as CpR where R is a non-natural guanosine nucleotide. Other preferred combinations comprise a saponin (in particular QS21) and a Toll like receptor 4 ligand such as monophosphoryl lipid A or its 3 deacylated derivative, 3 D-MPL, or a saponin (in particular QS21) and a Toll like receptor 4 ligand such as an alkyl glucosaminide phosphate. Other preferred combinations comprise a TLR 3 or 4 ligand in combination with a TLR 8 or 9 ligand. In one embodiment, the toll like receptor ligand is a receptor agonist. In another embodiment, the toll like receptor ligand is a receptor antagonist. The term “ligand” as used throughout the specification and the claims is intended to mean an entity that can bind to the receptor and have an effect, either to upregulate or downregulate the activity of the receptor.

Particularly preferred adjuvants are combinations of 3D-MPL and QS21 (EP 0 671 948 B1), oil in water emulsions comprising 3D-MPL and QS21 (WO 95/17210, WO 98/56414), or 3D-MPL formulated with other carriers (EP 0 689 454 B1). Other preferred adjuvant systems comprise a combination of 3 D MPL, QS21 and a CpG oligonucleotide as described in U.S. Pat. No. 6,558,670, U.S. Pat. No. 6,544,518.

In an embodiment the adjuvant is a Toll like receptor (TLR) 4 ligand, preferably an ligand such as a lipid A derivative particularly monophosphoryl lipid A or more particularly 3 Deacylated monophoshoryl lipid A (3 D-MPL).

3 D-MPL is sold under the trademark MPL® by Corixa corporation and primarily promotes CD4+T cell responses with an IFN-g (Th1) phenotype. It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3 D-MPL is used. Small particle 3 D-MPL has a particle size such that it may be sterile-filtered through a 0.22 μm filter. Such preparations are described in International Patent Application No. WO 94/21292. Synthetic derivatives of lipid A are known and thought to be TLR 4 ligands including, but not limited to:

OM174 (2-deoxy-6-o[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)

OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462)

OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Another preferred immunostimulant for use in the present invention is Quil A and its derivatives. Quil A is a saponin preparation isolated from the South American tree Quilaja Saponaria Molina and was first described as having adjuvant activity by Dalsgaard et al. in 1974 (“Saponin adjuvants”, Archiv. für die gesamte Virusforschung, Vol. 44, Springer Verlag, Berlin, p 243-254). Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina which induces CD8+ cytotoxic T cells (CTLs), Th1 cells and a predominant IgG2a antibody response and is a preferred saponin in the context of the present invention.

Particular formulations of QS21 have been described which are particularly preferred, these formulations further comprise a sterol (WO96/33739). The saponins forming part of the present invention may be separate in the form of micelles, mixed micelles (preferentially, but not exclusively with bile salts) or may be in the form of ISCOM matrices (EP 0 109 942 B1), liposomes or related colloidal structures such as worm-like or ring-like multimeric complexes or lipidic/layered structures and lamellae when formulated with cholesterol and lipid, or in the form of an oil in water emulsion (for example as in WO 95/17210). The saponins may preferably be associated with a metallic salt, such as aluminium hydroxide or aluminium phosphate (WO 98/15287). Preferably, the saponin is presented in the form of a liposome, ISCOM or an oil in water emulsion.

Immunostimulatory oligonucleotides or any other Toll-like receptor (TLR) 9 ligand may also be used. The preferred oligonucleotides for use in adjuvants or vaccines of the present invention are CpG containing oligonucleotides, preferably containing two or more dinucleotide CpG motifs separated by at least three, more preferably at least six or more nucleotides. A CpG motif is a Cytosine nucleotide followed by a Guanine nucleotide. The CpG oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204.

Examples of preferred oligonucleotides have the following sequences. The sequences preferably contain phosphorothioate modified internucleotide linkages.

| | |
|---|---|
| OLIGO 1 (SEQ ID NO: 1): | TCC ATG ACG TTC CTG ACG TT |
| | (CpG 1826) |
| OLIGO 2 (SEQ ID NO: 2): | TCT CCC AGC GTG CGC CAT |
| | (CpG 1758) |
| OLIGO 3 (SEQ ID NO: 3): | ACC GAT GAC GTC GCC GGT |
| | GAC GGC ACC ACG |
| OLIGO 4 (SEQ ID NO: 4): | TCG TCG TTT TGT CGT TTT |
| | GTC GTT (CpG 2006) |
| OLIGO 5 (SEQ ID NO: 5): | TCC ATG ACG TTC CTG ATG |
| | CT (CpG 1668) |
| OLIGO 6 (SEQ ID NO: 6): | TCG ACG TTT TCG GCG CGC |
| | GCC G (CpG 5456) |

Alternative CpG oligonucleotides may comprise the preferred sequences above in that they have inconsequential deletions or additions thereto.

Alternative immunostimulatory oligonucleotides may comprise modifications to the nucleotides. For example, WO0226757 and WO03507822 disclose modifications to the C and G portion of a CpG containing immunostimulatory oligonucleotides.

The immunostimulatory oligonucleotides utilised in the present invention may be synthesized by any method known in the art (for example see EP 468520). Conveniently, such oligonucleotides may be synthesized utilising an automated synthesizer.

Examples of a TLR 2 ligand include peptidoglycan or lipoprotein. Imidazoquinolines, such as Imiquimod and Resiquimod are known TLR7 ligands. Single stranded RNA is also a known TLR ligand (TLR8 in humans and TLR7 in mice), whereas double stranded RNA and poly IC (polyinosinic-polycytidylic acid—a commercial synthetic mimetic of viral RNA). are exemplary of TLR 3 ligands. 3D-MPL is an example of a TLR4 ligand whilst CPG is an example of a TLR9 ligand.

In one embodiment the B subunit of Shiga toxin or immunologically functional equivalent thereof and the first antigen are complexed together. By complexed is meant that the B subunit of Shiga toxin or immunologically functional equivalent thereof and the antigen are physically associated, for example via an electrostatic or hydrophobic interaction or a covalent linkage. In a preferred embodiment the B subunit of Shiga toxin and antigen are covalently linked either as a fusion protein (Haicheur et al, 2000 Journal of Immunology 165 pp 3301-3308) or linked via a cysteine residue in the manner as described in WO02/060937 (supra). In embodiments of the invention more than one antigen is linked to each toxin B molecule, such as 2, 3, 4, 5 6 antigen molecules per toxin B. When more than one antigen is linked to each toxin B molecule, these antigens may all be the same, one or more may be different to the others, or all the antigens may be different to each other.

The antigens themselves may be a peptide, or a protein encompassing one or more epitopes of interest. It is a preferred embodiment that the first antigen is selected such that when complexed with a protein of the invention it provides immunity against intracellular pathogens such as HIV, tuberculosis, Chlamydia, HBV, HCV, and Influenza. The present Invention also finds utility with antigens which can raise relevant immune responses against benign and proliferative disorders such as Cancers.

Preferably the vaccine formulations of the present invention contain an antigen or antigenic composition capable of eliciting an immune response against a human pathogen, which antigen or antigenic composition is derived from HIV-1, (such as gag or fragments thereof, such as p24, tat, nef, envelope such as gp120 or gp160, or fragments of any of these), human herpes viruses, such as gD or derivatives thereof or Immediate Early protein such as ICP27 from HSV1 or HSV2, cytomegalovirus ((esp Human) (such as gB or derivatives thereof), Rotaviral antigen, Epstein Barr virus (such as gp350 or derivatives thereof), Varicella Zoster Virus (such as gpI, II and IE63), or from a hepatitis virus such as hepatitis B virus (for example Hepatitis B Surface antigen or a derivative thereof), or antigens from hepatitis A virus, hepatitis C virus and hepatitis E virus, or from other viral pathogens, such as paramyxoviruses: Respiratory Syncytial virus (such as F G and N proteins or derivatives thereof), parainfluenza virus, measles virus, mumps virus, human papilloma viruses (for example HPV 6, 11, 16, 18,) flaviviruses (e.g. Yellow Fever Virus, Dengue Virus, Tick-borne encephalitis virus, Japanese Encephalitis Virus) or Influenza virus purified or recombinant proteins thereof, such as HA, NP, NA, or M proteins, or combinations thereof), or derived from bacterial pathogens such as Neisseria spp, including N. gonorrhea and N. meningitidis (for example, transferrin-binding proteins, lactoferrin binding proteins, PiIC, adhesins); S. pyogenes (for example M proteins or fragments thereof, C5A protease,), S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Most preferred antigens for Chlamydia include for example the High Molecular Weight Protein (HMW) (WO 99/17741), ORF3 (EP 366 412), and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (for example, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B, non typeable H. influenzae, for example OMP26, high molecular weight adhesins, P5, P6, protein D and lipoprotein D, and fimbrin and fimbrin derived peptides (U.S. Pat. No. 5,843,464) or multiple copy varients or fusion proteins thereof.

Derivatives of Hepatitis B Surface antigen are well known in the art and include, inter alia, those PreS1, PreS2 S antigens set forth described in European Patent applications EP-A-414 374; EP-A-0304 578, and EP 198-474. In one preferred aspect the vaccine formulation of the invention comprises the HIV-1 antigen, gp120, especially when expressed in CHO cells. In a further embodiment, the vaccine formulation of the invention comprises gD2t as hereinabove defined.

In a preferred embodiment of the present invention vaccines containing the claimed adjuvant comprise antigen derived from the Human Papilloma Virus (HPV) considered to be responsible for genital warts (HPV 6 or HPV 11 and others), and the HPV viruses responsible for cervical cancer (HPV16, HPV18 and others).

Particularly preferred forms of genital wart prophylactic, or therapeutic, vaccine comprise L1 protein, and fusion proteins comprising one or more antigens selected from the HPV proteins E1, E2, E5, E6, E7, L1, and L2.

The most preferred forms of fusion protein are: L2E7 as disclosed in WO 96/26277, and protein D(1/3)-E7 disclosed in WO99/10375.

A preferred HPV cervical infection or cancer, prophylaxis or therapeutic vaccine, composition may comprise HPV 16 or 18 antigens.

Particularly preferred HPV 16 antigens comprise the early proteins E6 or E7 in fusion with a protein D carrier to form Protein D-E6 or E7 fusions from HPV 16, or combinations thereof; or combinations of E6 or E7 with L2 (WO 96/26277).

Alternatively the HPV 16 or 18 early proteins E6 and E7, may be presented in a single molecule, preferably a Protein D-E6/E7 fusion. Such vaccine may optionally contain either or both E6 and E7 proteins from HPV 18, preferably in the form of a Protein D-E6 or Protein D-E7 fusion protein or Protein D E6/E7 fusion protein.

The vaccine of the present invention may additionally comprise antigens from other HPV strains, preferably from strains HPV 31 or 33.

Vaccines of the present invention further comprise antigens derived from parasites that cause Malaria, for example, antigens from Plasmodia falciparum including circumsporozoite protein (CS protein), RTS,S, MSP1, MSP3, LSA1, LSA3, AMA1 and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in International Patent Application No. PCT/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in International Patent Application No. PCT/GB89/00895, published under WO 90/01496. Plasmodia antigens that are likely candidates to be components of a multistage Malaria vaccine are P. falciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp. One embodiment of the present invention is a malaria vaccine wherein the antigen preparation comprises RTS,S or CS protein or a fragment thereof such as the CS portion of RTS,S, in combination with one or more further malarial antigens, either or both of which may be attached to the Shiga toxin B subunit in accordance with the invention. The one or more further malarial antigens may be selected for example from the group consisting of MPS1, MSP3, AMA1, LSA1 or LSA3.

The formulations may also contain an anti-tumour antigen and be useful for the immunotherapeutic treatment of cancers. For example, the adjuvant formulation finds utility with tumour rejection antigens such as those for prostrate, breast, colorectal, lung, pancreatic, renal or melanoma cancers. Exemplary antigens include MAGE 1 and MAGE 3 or other MAGE antigens (for the treatment of melanoma), PRAME, BAGE, or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p 293. Indeed these antigens are expressed in a wide range of tumour types such as melanoma, lung carcinoma, sarcoma and bladder carcinoma. Other tumour-specific antigens are suitable for use with the adjuvants of the present invention and include, but are not restricted to tumour-specific gangliosides, Prostate specific antigen (PSA) or Her-2/neu, KSA (GA733), PAP, mammaglobin, MUC-1, carcinoembryonic antigen (CEA), p501S (prostein). Accordingly in one aspect of the present invention there is provided a vaccine comprising an adjuvant composition according to the invention and a tumour rejection antigen. In one aspect, the tumour antigen is Her-2/neu.

It is a particularly preferred aspect of the present invention that the vaccines comprise a tumour antigen such as prostrate, breast, colorectal, lung, pancreatic, renal, ovarian or melanoma cancers. Accordingly, the formulations may contain tumour-associated antigen, as well as antigens associated with tumour-support mechanisms (e.g. angiogenesis, tumour invasion). Additionally, antigens particularly relevant for vaccines in the therapy of cancer also comprise Prostate-specific membrane antigen (PSMA), Prostate Stem Cell Antigen (PSCA), p501S (prostein), tyrosinase, survivin, NY-ESO1, prostase, PS108 (WO 98/50567), RAGE, LACE, HAGE. Additionally said antigen may be a self peptide hormone such as whole length Gonadotrophin hormone releasing hormone (GnRH, WO 95/20600), a short 10 amino acid long peptide, useful in the treatment of many cancers, or in immunocastration.

Vaccines of the present invention may be used for the prophylaxis or therapy of allergy. Such vaccines would comprise allergen specific antigens, for example Der p1

In one aspect of the invention, the vaccine compositions of the invention comprise more than one different antigen, wherein at least one antigen is complexed to a protein of the invention. Such compositions would be useful to raise immune responses wherein the antigen that is complexed to the protein of the invention is an internal antigen from a pathogen and as such needs to be directed into the MHC class I presenting pathway. In addition, the composition further comprises at least one second antigen that is not complexed to a protein of the invention. In a preferred aspect, this second non complexed antigen can raise an antibody response or can be directed through the MHC class II presenting pathway. This dual approach ensures that as many different arms of the immune system are stimulated as possible, thereby making it more likely that a protective immune response will be generated.

It is thought that such an approach will be particularly useful in generating an immune response against at least two antigens wherein the antigens not complexed to a protein of the invention are external pathogenic antigens (in other words, antigens substantially exposed on the outside of a pathogen and which are generally ‘visible’ to the immune system), for example the HPV L1 and L2 proteins, the Hepatitis C E1 protein, influenza virus HA or NA, the RSV F, G or SH proteins, the HBV HBs protein, the HIV gp120 protein, Dengue virus E protein, VZV gE protein, CMV gB protein and EBV gp350 protein, or immunogenic fragments thereof whilst the antigen or antigens complexed to a protein of the invention are internal pathogenic antigens. Examples of the latter include the HPV E1, E2, E3, E4, E5, E6, E7, E8, E9 antigens, the HCV NS1, NS2, NS3, NS4a, 4b, NS5a, 5b proteins, the influenza virus matrix, nucleoprotein, PB1, PB2, PA, NS2 or NS1 protein, RSV M1, M2-1, M2-2, L, NS1, NS2, or P protein or nucleoprotein, Hepatitis B virus HB core protein, HIV Nef, tat, P27, F4 or P24 protein, CMV pp 65 protein or Epstein Barr Virus latency related gene, or immunogenic fragments thereof. In one embodiment of the invention the antigens are from two different pathogens, whilst in another embodiment of the invention the antigens are from the same pathogen. In another embodiment of the invention the complexed antigen and the free antigen are the same. In this embodiment one advantage provided by the invention is the provision of CD8 and CD4 responses to the same antigen.

In one embodiment of the invention there are only two antigens present, one of which is not complexed with the protein of the invention, and one of which is complexed with a protein of the invention. In a further embodiment, there is only one antigen complexed to the protein of the invention, but the composition comprises more than one antigen which is not complexed to a protein of the invention. In a further embodiment, there are one or more antigens present which are not complexed to a protein of the invention, and more than one antigen present which is complexed to a protein of the invention. In this embodiment, each complexed antigen may be complexed to a separate protein of the invention, or more than one antigen for example 2, 3, 4 or 5 antigens may be complexed to one protein of the invention.

In a further embodiment, the composition may comprise, as well as a protein of the invention, a further protein as described in co-pending application UK 0524408.2, filed 30 Nov. 2005. This application describes similar compositions to those described therein, but the proteins of the invention in UK 0524408.2 are non-live vectors (excluding the Shiga toxin proteins described herein). The term “non-live vector” is defined as an antigen delivery agent which targets MHC class I presentation. This term is not intended to encompass replicating vectors, such as attenuated viruses, bacteria, or plasmid DNA. The non-live vector is derived from a bacterial toxin, that is the non-live vector is a detoxified bacterial toxin, subunit or immunologically functional equivalent.

In the context of the invention of UK 0524408.2, the word toxin is intended to mean toxins that have been detoxified such that they are no longer toxic to humans, or a toxin subunit or fragment thereof that are substantially devoid of toxic activity in humans.

Preferred non-live vectors based on detoxified toxins are the amino terminal domain of the anthrax lethal factor (LF), P. aeruginosa exotoxin A, the B subunit from E. coli labile toxin (LT), and the adenylate cyclase A from B pertussis. In one embodiment, the non-live vector is the B subunit from E. coli labile toxin type I (LTI) In one embodiment, the non-live vector is derived from a toxin which is a family of the AB5 family, for example LT2, the cholera toxin (CT), the Bordatella Pertussis toxin (PT) as well as the recently identified subtilase cytotoxins. (Paton et al, J Exp Med 2004, Vol 200 pp 35-46).

In this embodiment, the non-live vector based on a bacterial toxin or immunologically functional equivalent thereof is also used to complex an antigen. Thus, for example, a composition of the present invention, may comprise one or more free antigens, one or more antigens complexed with one or more proteins of the invention, and one or more antigens complexed with a non-live vector or an immunologically functional equivalent therefore as described in UK 0524408.2.

In one embodiment, the antigens are viral antigens. In one aspect suitable viral antigens for use either in complexing to the protein of the invention, or for use in an uncomplexed form, may be selected from the lists given above.

In one aspect the antigen not complexed to a protein of the invention is the HPV L1 protein or immunogenic fragment thereof. Suitable L1 proteins and L1 protein fragments are well known in the art, for example as disclosed in WO2004/056389 and references therein, all herein incorporated by reference. In one aspect the L1 protein is full length L1. In one aspect the L1 protein is a truncated L1 protein. In one aspect the L1 protein is in the form of a virus like particle (VLP), the VLP being made up of either full length or truncated L1. Where L1 is truncated, then in one aspect the truncation removes a nuclear localisation signal. In one aspect the truncation is a C terminal truncation. In one aspect the C terminal truncation removes less than 50 amino acids, for example less than 40 amino acids. Where the L1 is an HPV 16 VLP then in one aspect the C terminal truncation removes 34 amino acids from HPV 16 L1. Where the VLP is an HPV 18 VLP then in one aspect the C terminal truncation removes 35 amino acids from HPV 18 L1. L1 may be selected from any suitable HPV, for example oncogenic HPV types such as HPV 16, 18, 31, 33, 35, 39, 45, 51, 52, 56, 58, 59, 66, 68.

Truncated L1 proteins are suitably functional L1 protein derivatives. Functional L1 protein derivatives are capable of raising an immune response (if necessary, when suitably adjuvanted), said immune response being capable of recognising a VLP consisting of the full length L1 protein and/or the HPV type from which the L1 protein was derived.

Where one antigen not complexed to a protein of the invention is the HPV L1 protein or immunogenic fragment thereof, then in one aspect one antigen complexed to a protein of the invention is the HPV E2 protein, or E4 protein, or E5 protein, or E6 protein, or E7 protein, or immunogenic fragments thereof.

In one embodiment of the invention, the composition of the invention comprises HPV 16 L1 and HPV 18 L1 as free antigens, and one or more HPV early proteins as complexed antigen. Preferably early proteins are present from both HPV 16 and 18. Preferably more than one early protein is present. In one aspect of this embodiment, the composition comprises HPV16 E7 and HPV 18 E7. In a further particular aspect of this embodiment, the composition comprises HPV16 E2, HPV 18E2, HPV 16 E6 and HPV 18 E6 as complexed antigens. In one aspect of this embodiment, HPV 16 and HPV 18 L1 are present in the form of VLPs.

In one aspect one antigen not complexed to a protein of the invention is the HCV E1 protein or immunogenic fragment thereof, such as a truncate thereof, for example, a C terminal E1 truncate, and one antigen complexed to a protein of the invention is the HCV NS3 protein or immunogenic fragment thereof.

In one aspect one antigen not complexed to a protein of the invention is the VZv gE protein or immunogenic fragment thereof. One antigen complexed to a protein of the invention in this case may be, for example, IE63 or IE62, or immunogenic fragments thereof.

In one aspect one antigen not complexed to a protein of the invention is the HCMV gB protein or immunogenic fragment thereof, or the gH protein or immunogenic fragment thereof. In one aspect one antigen not complexed to a protein of the invention is the pp 65 protein or immunogenic fragment thereof, or the major immediate early protein IE1 72, or immunogenic fragment thereof.

In one aspect of the invention, one antigen not complexed to a protein of the invention is an influenza virus subunit antigen, for example NA or HA or immunogenic fragment thereof or combinations thereof. In a further aspect, an influenza split preparation may be used in the composition to provide the antigens not complexed to a protein of the invention. One antigen complexed to a protein of the invention in these cases may be, for example, influenza virus matrix protein, NP, PB1, PB2, PA, NS2 or NS1 protein or immunogenic fragments thereof.

In one aspect of the invention, one antigen not complexed to a protein of the invention is an RSV F, G or SH protein or immunogenic fragment thereof. In this case, one antigen complexed to a protein of the invention may be, for example, an RSV RSV M1, M2-1, M2-2, L, P, NS1, NS2, N protein or an immunogenic fragment thereof.

In one aspect of the invention, one antigen not complexed to a protein of the invention is an HBV HBs protein or an immunogenic fragment thereof. In this case, one antigen complexed to a protein of the invention may be, for example, HB core protein or an immunogenic fragment thereof.

In one aspect of the invention, one antigen not complexed to a protein of the invention is an HIV gp120 protein or an immunogenic fragment thereof. In this case, one antigen complexed to a protein of the invention may be, for example, an HIV Nef, tat, P27, F4 or P24 protein or an immunogenic fragment thereof.

In one aspect of the invention, one antigen not complexed to a protein of the invention is a Dengue virus E protein or an immunogenic fragment thereof. In this case, one antigen complexed to a protein of the invention may be, for example, a dengue virus NS1 protein or an immunogenic fragment thereof.

In one aspect of the invention, one antigen not complexed to a protein of the invention is an EBV gp350 protein or an immunogenic fragment thereof. In this case, one antigen complexed to a protein of the invention may be, for example, an EBV latency related gene product or an immunogenic fragment thereof.

Example of immunogenic fragments of antigens include, for example, peptides comprising B and/or T cell epitopes, and which can be used to stimulate an immune response.

Where 2 different antigens are used from the same virus, such as HPV L1 and HPV E5, then in one aspect the antigens are from the same viral type or subtype—e.g. both from HPV 16. This principle can be applied to antigen combinations from other viruses.

In a further aspect of the invention, the vaccine compositions of the invention comprise an antigen complexed to a protein of the invention, and further comprise the same antigen as free antigen, i.e. not complexed to a protein of the invention

In all of the above described aspects of the invention, the vaccine composition further comprises an adjuvant as described herein.

The amount of each antigen in each vaccine dose is selected as an amount which induces an immunoprotective response without significant, adverse side effects in typical vaccinees. Such amount will vary depending upon which specific immunogen is employed and how it is presented. Where a composition comprises a metal salt as sole adjuvant, it will be appreciated by a person skilled in the art that the level of free antigen (as measured by, for example, the method set out in example 1.5) will be the determinative amount for immunoprotection.

Generally, it is expected that each human dose will comprise 0.1-1000 μg of each antigen, preferably 0.1-500 μg, preferably 0.1-100 μg, most preferably 0.1 to 50 μg. An optimal amount for a particular vaccine can be ascertained by standard studies involving observation of appropriate immune responses in vaccinated subjects. Following an initial vaccination, subjects may receive one or several booster immunisation adequately spaced. Such a vaccine formulation may be applied to a mucosal surface of a mammal in either a priming or boosting vaccination regime; or alternatively be administered systemically, for example via the transdermal, subcutaneous or intramuscular routes. Intramuscular administration is preferred.

The amount of 3 D MPL used is generally small, but depending on the vaccine formulation may be in the region of 1-1000 μg per dose, preferably 1-500 μg per dose, and more preferably between 1 to 100 μg per dose.

The amount of CpG or immunostimulatory oligonucleotides in the adjuvants or vaccines of the present invention is generally small, but depending on the vaccine formulation may be in the region of 1-1000 μg per dose, preferably 1-500 μg per dose, and more preferably between 1 to 100 μg per dose.

The amount of saponin for use in the adjuvants of the present invention may be in the region of 1-1000 μg per dose, preferably 1-500 μg per dose, more preferably 1-250 μg per dose, and most preferably between 1 to 100 μg per dose.

The formulations of the present invention maybe used for both prophylactic and therapeutic purposes. Accordingly the invention provides a vaccine composition as described herein for use in medicine.

In a further embodiment there is provided a method of treatment of an individual susceptible to or suffering from a disease by the administration of a composition as substantially described herein.

Also provided is a method to prevent an individual from contracting a disease selected from the group comprising infectious bacterial and viral diseases, parasitic diseases, particularly intracellular pathogenic disease, proliferative diseases such as prostate, breast, colorectal, lung, pancreatic, renal, ovarian or melanoma cancers; non-cancer chronic disorders, allergy comprising the administration of a composition as substantially described herein to said individual.

Furthermore, there is described a method of inducing a CD8+ antigen specific immune response in a mammal, comprising administering to said mammal a composition of the invention. Further there is provided a method of manufacture of a vaccine comprising admixing an antigen in combination with the B subunit of shiga toxin or immunological functional equivalent thereof is admixed with an adjuvant.

Examples of suitable pharmaceutically acceptable excipients for use in the combinations of the present invention include, among others, water, phosphate buffered saline, isotonic buffer solutions

All publications, including but not limited to patents and patent applications, cited in this specification are herein incorporated by reference as if each individual publication were specifically and individually indicated to be incorporated by reference herein as though fully set forth.

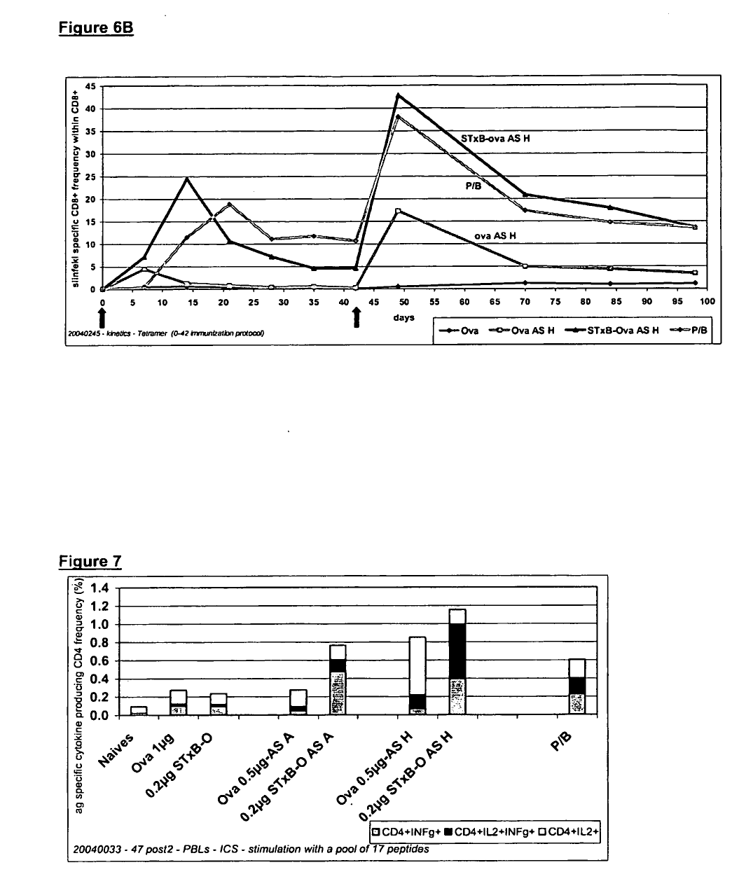

The present invention is exemplified by reference to the following examples and figures. In all figures, adeno-ova (adenovirus vector containing OVA protein) was used as a positive control in first injection. P/B (prime/boost) is a positive control with first injection of Adeno-Ova, and second, boost injection of Ova in AS A. (AS H in FIG. 6B):

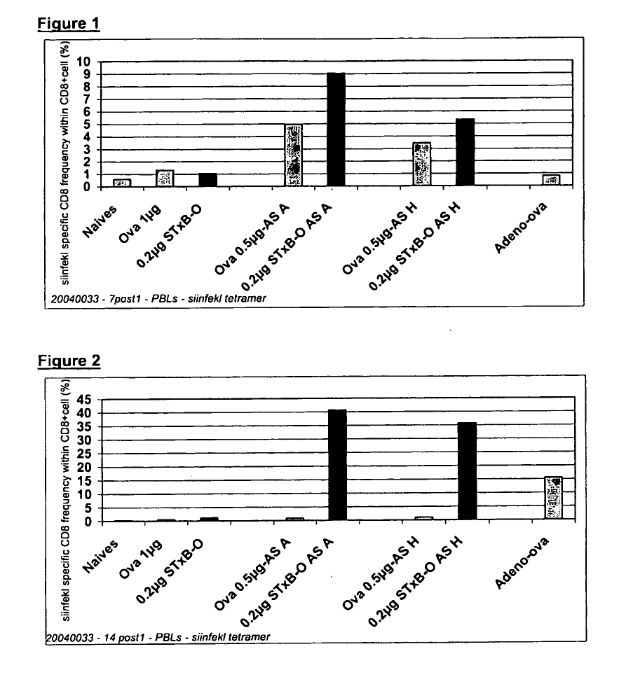

FIG. 1: Siinfekl-specific CD 8 frequency in PBLs 7 days after primary injection with AS A STxB Ova and AS H STxB Ova vaccines.

FIG. 2 Siinfekl-specific CD 8 frequency in PBLs 14 days after primary injection with AS A STxB Ova and AS H STxB Ova vaccines.

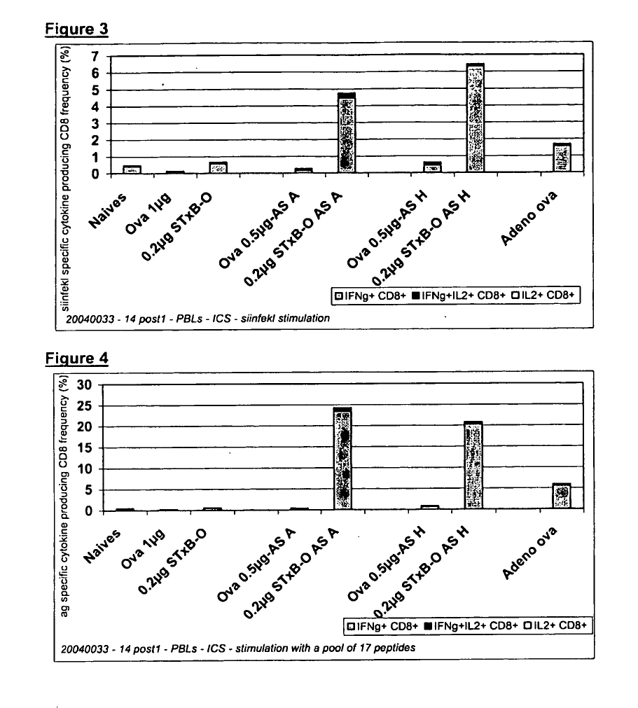

FIG. 3 Effector T cell response persistency assessed in PBLs through siinfekl-specific cytokine-producing CD8 T cells at day 15 after primary injection with AS A STxB Ova and AS H STxB Ova vaccines.

FIG. 4 Effector T cell response persistency assessed in PBLs through antigen-specific cytokine-producing CD8 T cells at day 15 after primary injection with AS A STxB Ova and AS H STxB Ova vaccines.

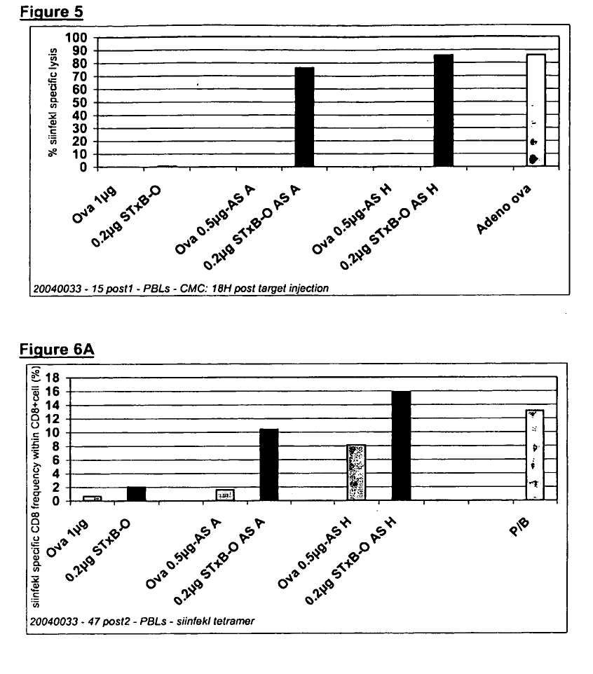

FIG. 5 Effector T cell response assessed by cytotoxic activity detected in vivo 15 days after primary injection with AS A STxB Ova and AS H STxB Ova vaccines.

FIG. 6: (A) Siinfekl-specific CD8 frequency in PBLs 47 days after second injection with AS A STxB Ova and AS H STxB Ova vaccines. (B) Kinetics of the Siinfekl-specific CD8 frequency in PBLs from day 0 to day 98.

FIG. 7: Effector T cell response assessed through antigen-specific cytokine-producing CD4 T cells in PBLs 47 days after second injection with AS A and AS H STxB Ova vaccines.

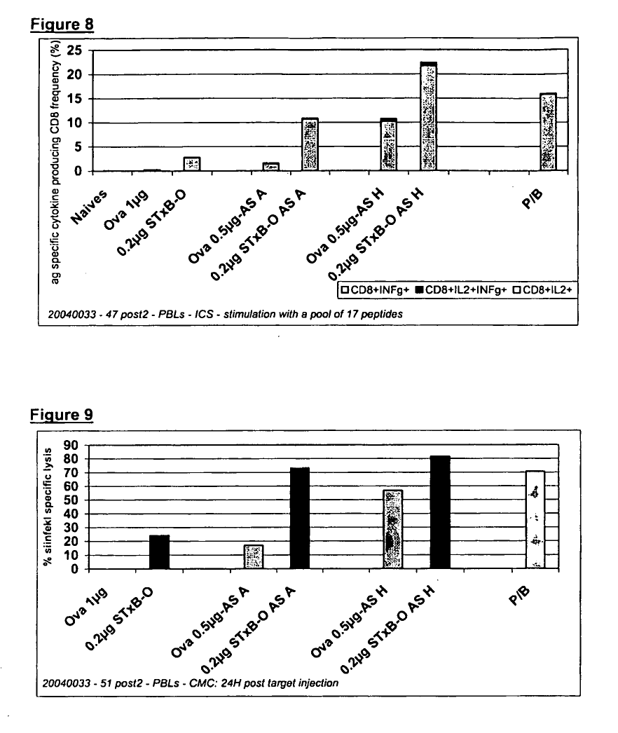

FIG. 8: Effector T cell response assessed through antigen-specific cytokine-producing CD8 T cells in PBLs 47 days after second injection with AS A and AS H STxB Ova vaccines.

FIG. 9: Effector T cell response assessed by Cytotoxic activity detected in vivo 47 days after second Injection with AS A STxB Ova and AS H STxB Ova vaccines.

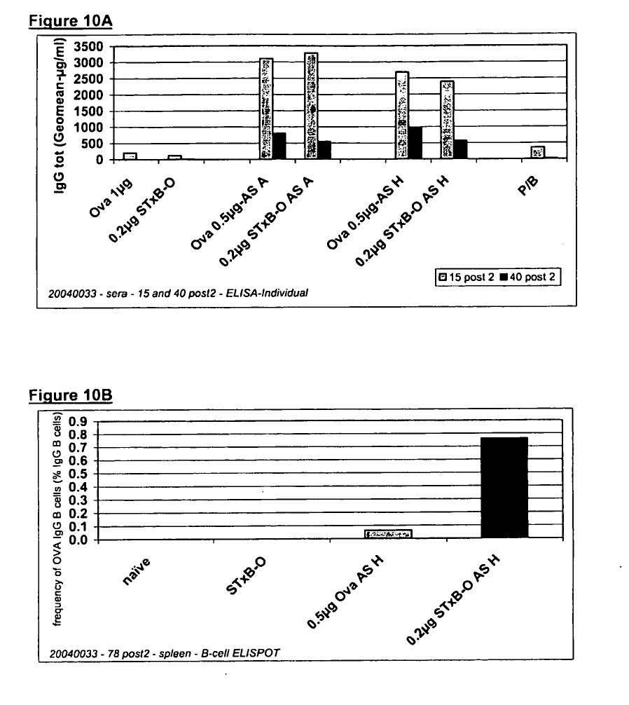

FIG. 10A: Humoral response 15 days and 40 days post second injection with AS A STxB Ova and AS H STxB Ova vaccines.

FIG. 10B: Anti-Ova memory B cells frequency assessed in spleen 78 days after the second injection of ASH STxB-OVA.

FIG. 11: Siinfekl-specific CD8 frequency in PBLs with AS A, AS F, AS D, AS E, STxB-ova vaccines 13 days post primary injection.

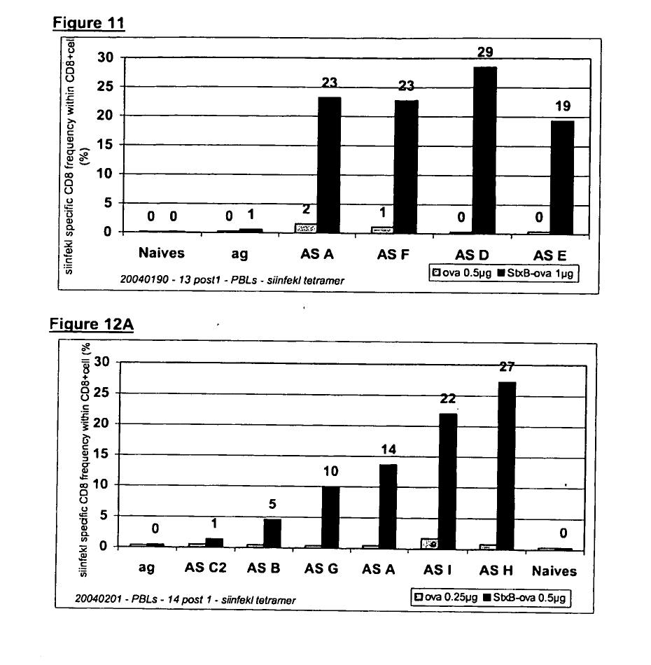

FIG. 12A: Siinfekl-specific CD8 frequency in PBLs with AS A, AS B, AS C, AS G, AS I, and AS H STxB-ova vaccines, 15 days post first injection.

FIG. 12 B: Siinfekl-specific CD8 frequency in PBLs with AS A, AS B, AS C, AS G, AS I, and AS H STxB-ova vaccines 6 days post second injection.

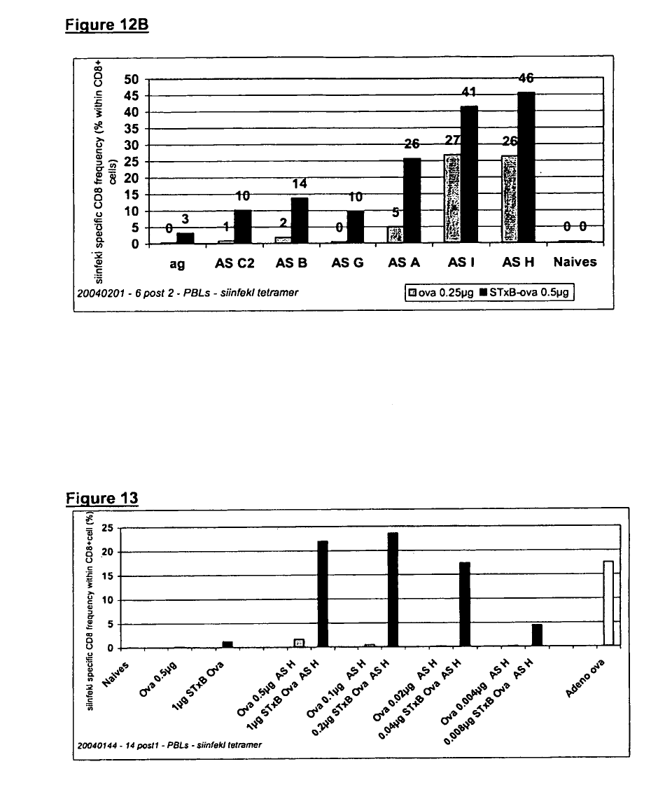

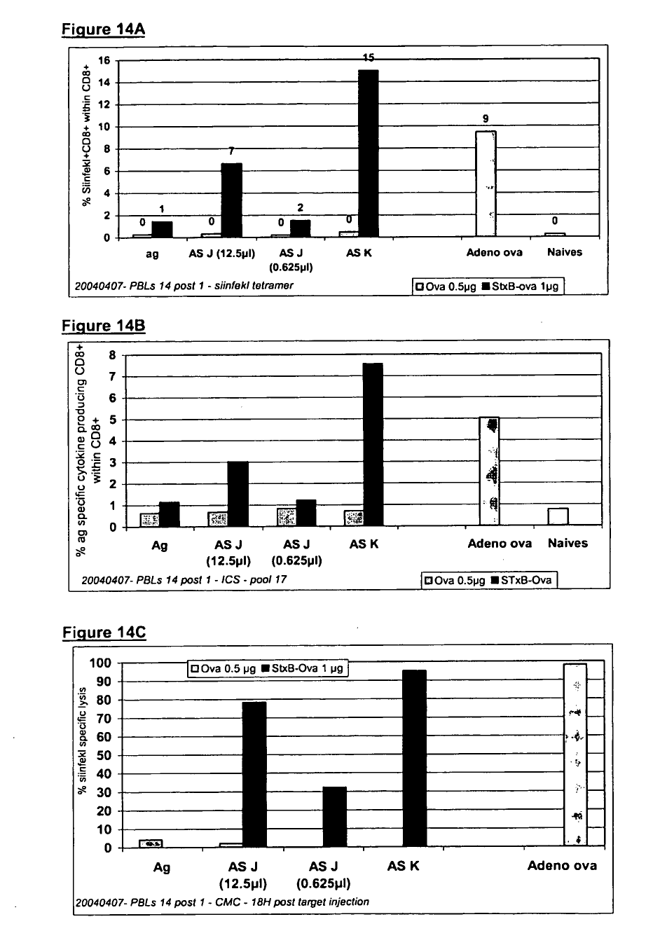

FIG. 13: Siinfekl-specific CD8 frequency in PBLs for different doses of STxB-ova vaccines formulated with the same dose of AS H.

FIG. 14: Evaluation of the immune response induced in vivo by STxB-ova with AS J (two doses) or AS K measured in PBLs 14 days after first injection. (A) Siinfekl-specific CD8 frequency. (B) antigen-specific cytokine-producing CD8 frequency. (C) Siinfekl-specific lysis detected in vivo

FIG. 15: Siinfekl-specific CD8 frequency in PBLs with AS L, AS G, AS M STxB-ova vaccines 14 days post 1^{st} injection.

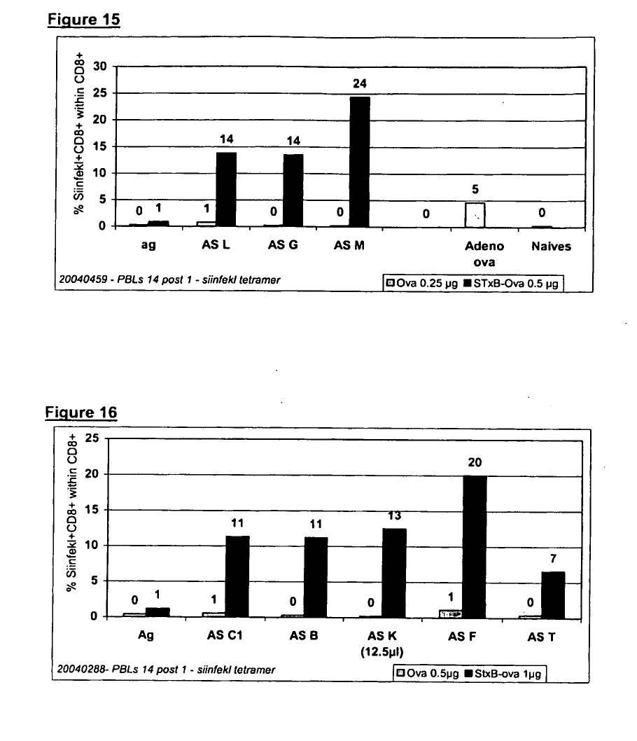

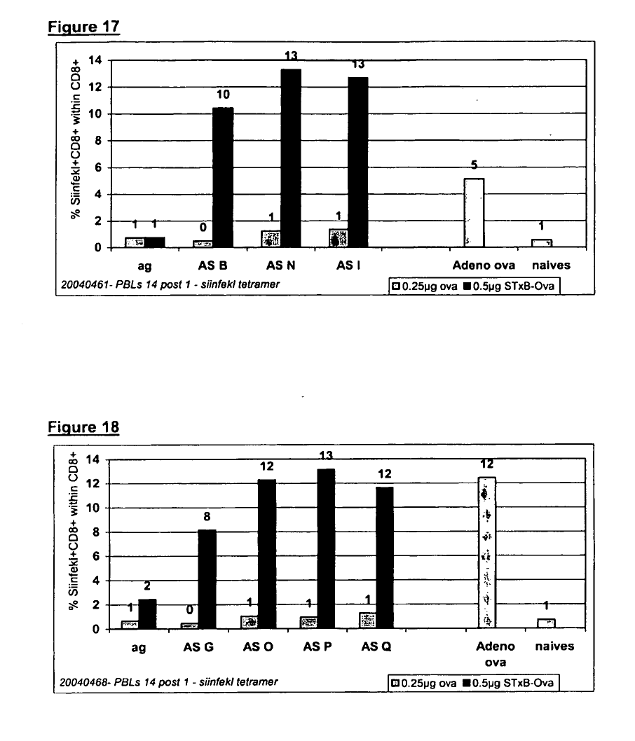

FIG. 16: Siinfekl-specific CD8 frequency in PBLs with AS B, AS C, AS K, AS F or AS T STxB ova vaccines 14 days post 1^{st} injection.

FIG. 17: Siinfekl-specific CD8 frequency in PBLs with AS B, AS N, AS I STxB-ova vaccines 14 days post 1^{st} injection.

FIG. 18: Siinfekl-specific CD8 frequency in PBLs 14 days post 1^{st} injection with AS G, AS O, AS P, AS Q STxB-ova vaccines.

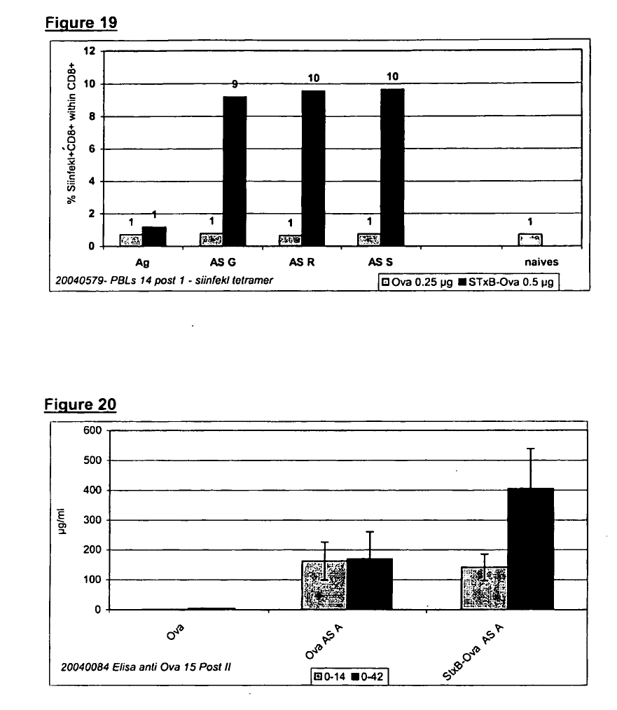

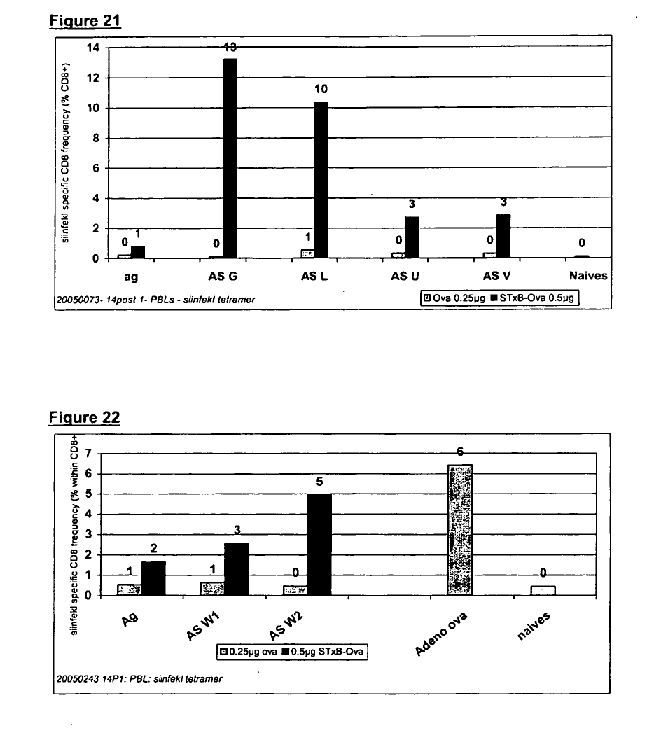

FIG. 19: Siinfekl-specific CD8 frequency in PBLs 14 days post 1^{st} injection with AS G, AS R, AS S STxB-ova vaccines.

FIG. 20: Humoral response detected 15 days after the second injection performed either 14 or 42 days after the first injection with AS A StxB-ova vaccine.

FIG. 21: Siinfekl-specific CD8 frequency in PBLs 14 days post 1^{st} injection with AS G, AS L, AS U, AS V STxB-ova vaccines.

FIG. 22: Siinfekl-specific CD8 frequency in PBLs 14 days post 1^{st} injection with ASW1, ASW2-ova vaccines.

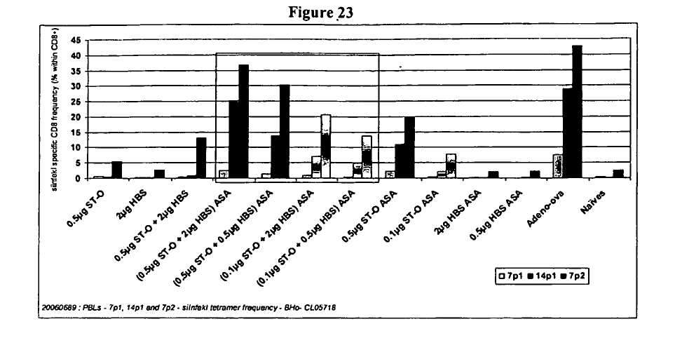

FIG. 23: Siinfekl-specific CD8 frequency in PBLs following injection with ASA adjuvanted composition comprising STx-Ova and HBs as free antigen, showing tetramer responses against Ova 7 post 1, 14 post 1 and 7 post 2.

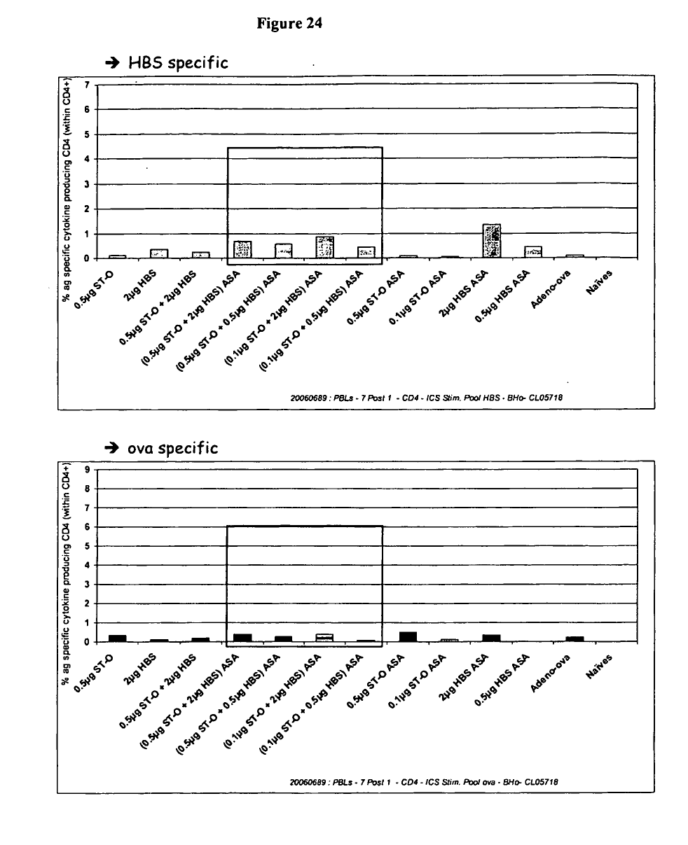

FIG. 24: % antigen specific cytokine producing CD4 frequency in PBLs 7 days post 1^{st} injection with ASA adjuvanted composition comprising STx-Ova and HBs as free antigen, top graph showing HBs responses, bottom graph showing Ova responses.

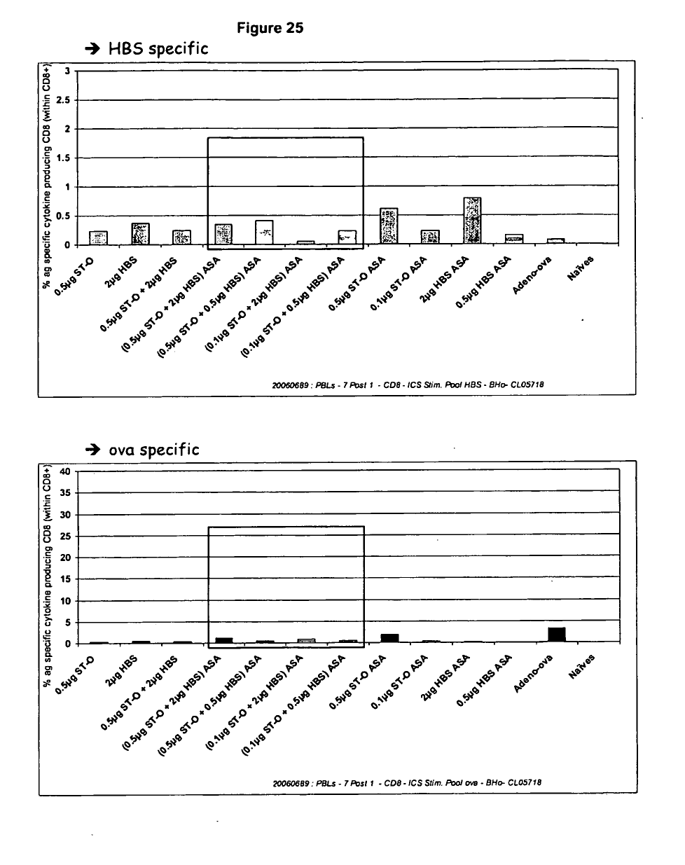

FIG. 25: % antigen specific cytokine producing CD8 frequency in PBLs 7 days post 1^{st} injection with ASA adjuvanted composition comprising STx-Ova and HBs as free antigen, top graph showing HBs responses, bottom graph showing Ova responses.

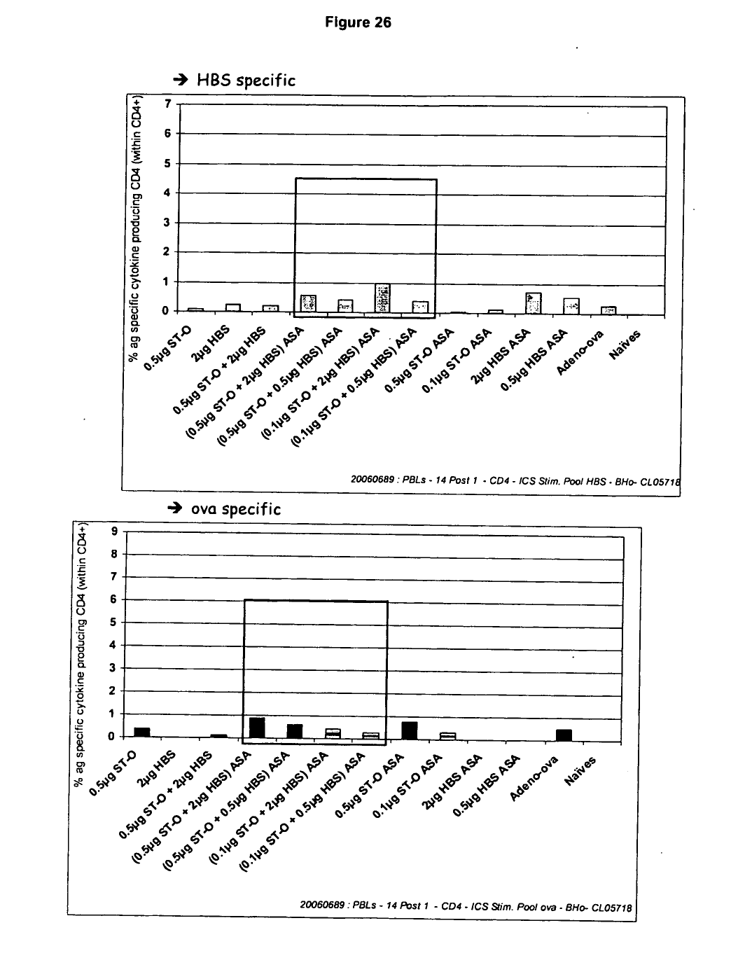

FIG. 26: % antigen specific cytokine producing CD4 frequency in PBLs 14 days post 1^{st} injection with ASA adjuvanted composition comprising STx-Ova and HBs as free antigen, top graph showing HBs responses, bottom graph showing Ova responses.

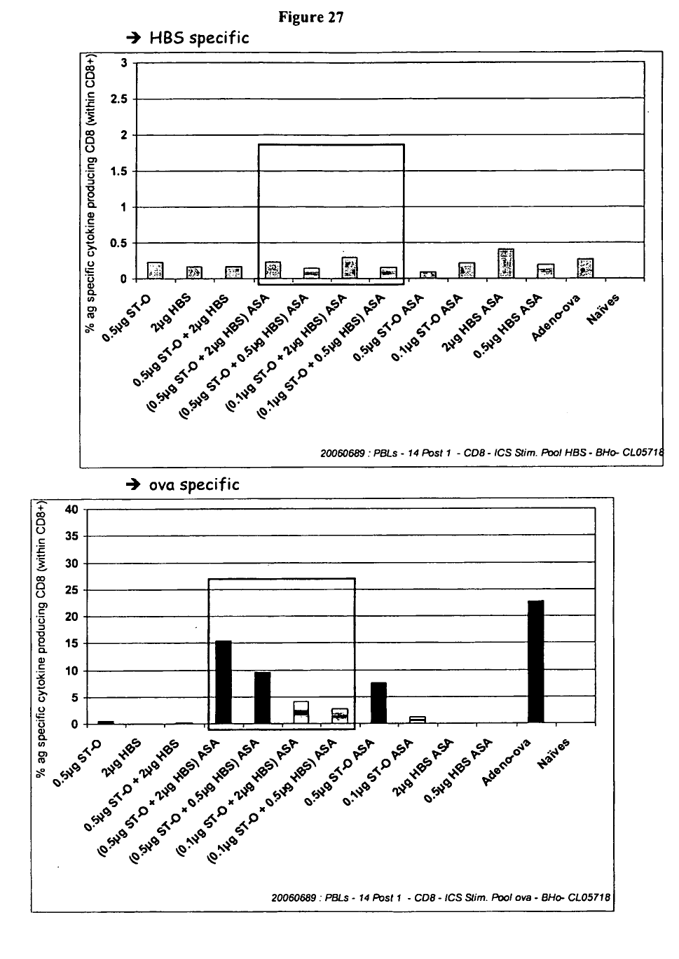

FIG. 27: % antigen specific cytokine producing CD8 frequency in PBLs 14 days post 1^{st} injection with ASA adjuvanted composition comprising STx-Ova and HBs as free antigen, top graph showing HBs responses, bottom graph showing Ova responses.

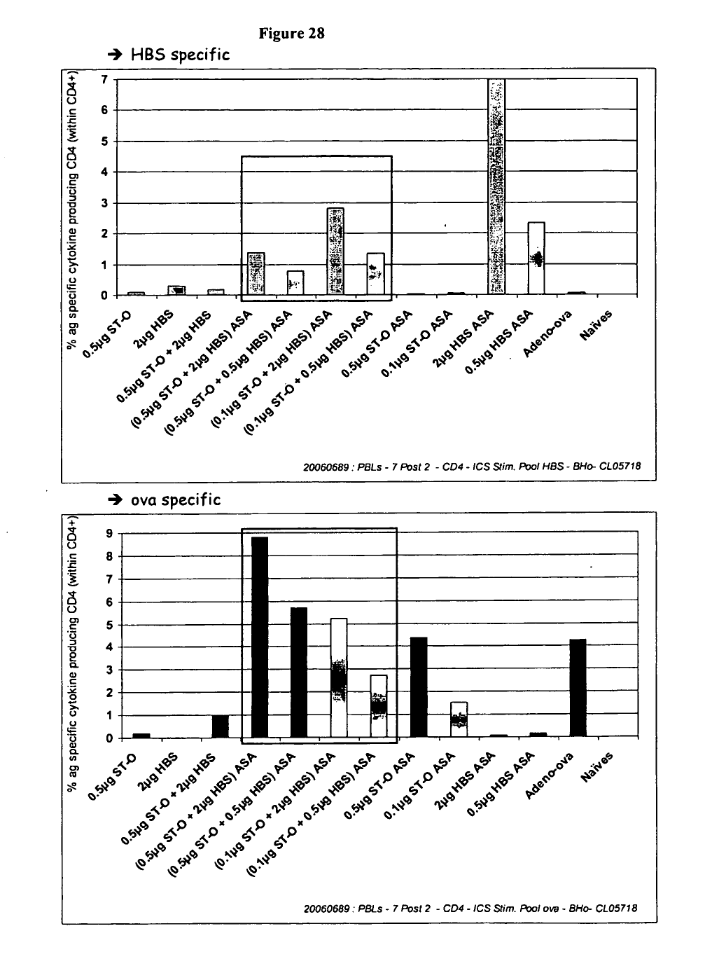

FIG. 28: % antigen specific cytokine producing CD4 frequency in PBLs 7 days post 2^{nd} injection with ASA adjuvanted composition comprising STx-Ova and HBs as free antigen, top graph showing HBs responses, bottom graph showing Ova responses.

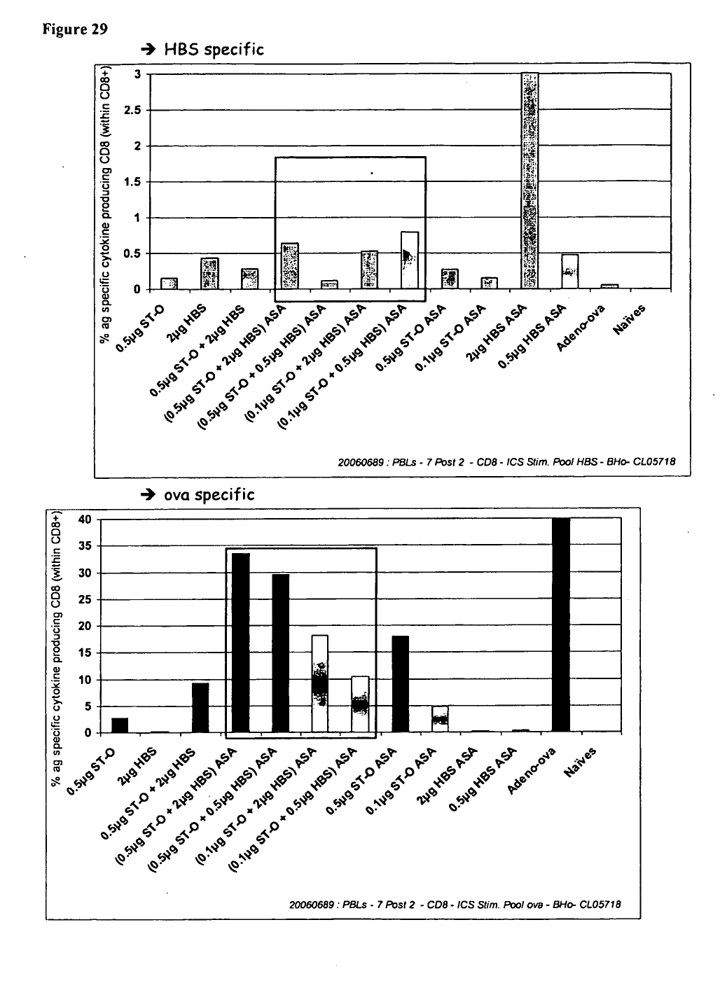

FIG. 29: % antigen specific cytokine producing CD8 frequency in PBLs 7 days post 2^{nd} injection with ASA adjuvanted composition comprising STx-Ova and HBs as free antigen, top graph showing HBs responses, bottom graph showing Ova responses.

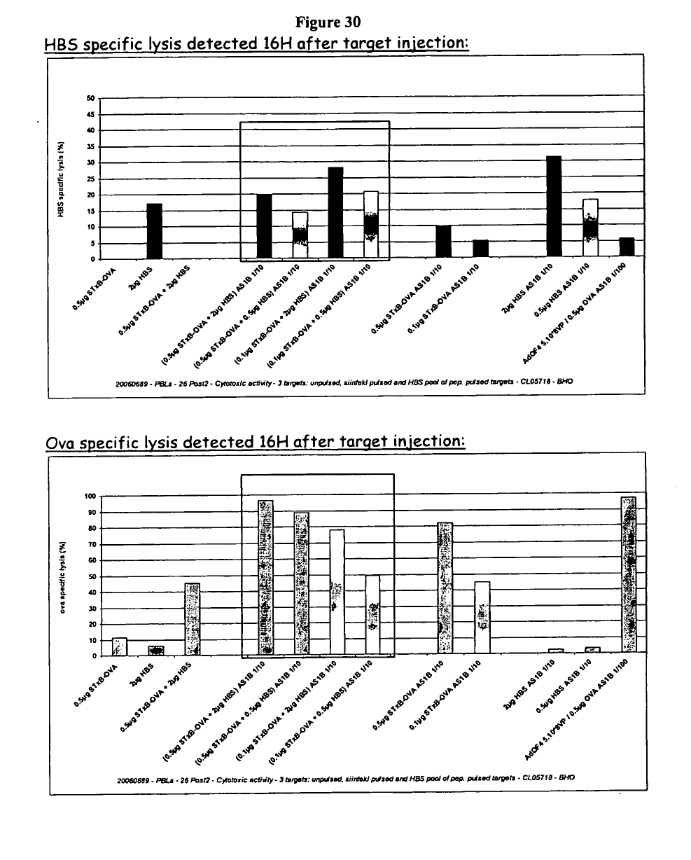

FIG. 30: Antigen specific lysis detected 16 hours after target injection, top graph showing HBs responses, bottom graph showing Ova responses.

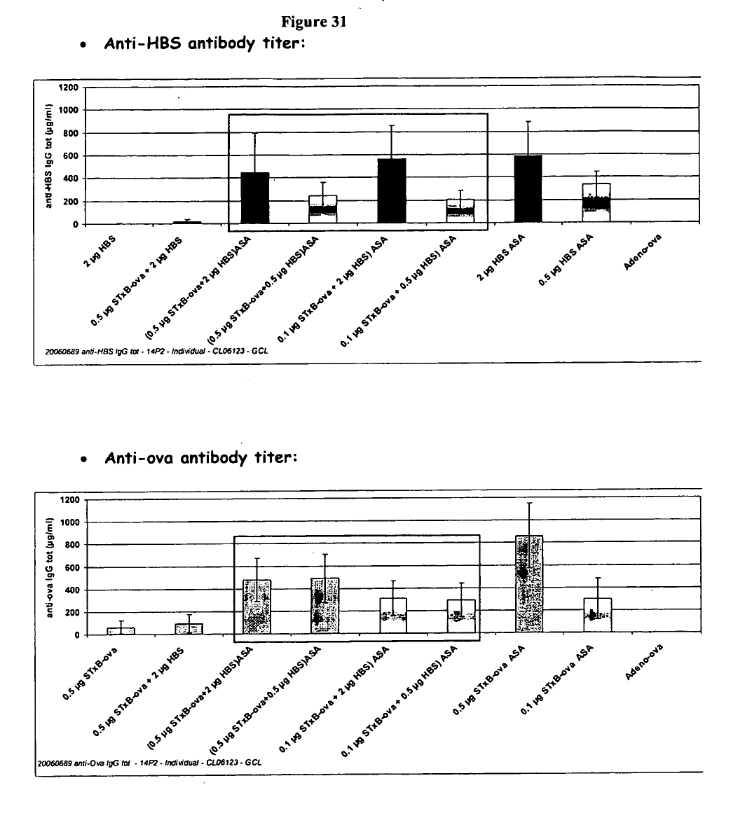

FIG. 31: Antibody responses against HBs (top) and Ova (bottom) 14 days post 2^{nd} injection with an ASA adjuvanted composition containing Stx-Ova and HBs as free antigen.

### EXAMPLES

### 1. Reagents and Media

1.1 Preparation of Adjuvanted STxB-Ova

STxB coupled to full length Chicken ovalbumin: to allow the chemical coupling of proteins to a defined acceptor site in STxB, a cysteine was added to the C-terminus of the wild-type protein, yielding STxB-Cys. The recombinant mutant STxB-Cys protein was produced as previously described (Haicheur et al.; 2000, J. Immunol. 165, 3301). Endotoxin concentration determined by the Limulus assay test was below 0.5 EU/ml. STxB-ova has been previously described (HAICHEUR et al., 2003, Int. Immunol., 15, 1161-1171) and was kindly provided by Ludger Johannes and Eric Tartour (Curie Institute).

StxB coupled to full length chicken ovalbumin was formulated in each of the adjuvant systems noted below.

1.2 Galabiose Binding Assay

The Gb3 receptor preferentially recognized by the B subunit of Shiga toxin is a cell surface glycosphingolipid, globotriaosylceramide (Galα1-4Galβ-4 glucosylceramide), where Gal is Galactose. The method described below is based on that described by Tarrago-Trani (Protein Extraction and Purification 38, pp 170-176, 2004), and involves an affinity chromatography on a commercially available galabiose-linked agarose gel (calbiochem). Galabiose (Galα1->4Gal) is the terminal carbohydrate portion of the oligosacharide moiety of Gb3 and is thought to represent the minimal structure recognized by the B subunit of Shiga toxin. This method has been successfully used to purify Shiga toxin directly from E. coli lysate. Therefore it can be assumed that proteins that bind this moiety will bind the Gb3 receptor.

The protein of interest in PBS buffer (500 μl) is mixed with 100 μl of immobilised galabiose resin (Calbiochem) previously equilibrated in the same buffer, and incubated for 30 min to 1 hour at 4° C. on a rotating wheel. After a first centrifugation at 5000 rpm for 1 min, the pellet is washed twice with PBS. The bound material is then eluated twice by re-suspending the final pellet in 2×500 μl of 100 mM glycine pH 2.5. Samples corresponding to the flow-through, the pooled washes and the pooled eluates are then analyzed by SDS Page, Coomassie staining and Western blotting. These analytical techniques allow identification of whether the protein is bound to the galabiose, and hence will bind the Gb3 receptor.

1.3—Preparation of Oil in Water Emulsion for Use in Adjuvant Systems.

Preparation of oil in water emulsion followed the protocol as set forth in WO 95/17210. The emulsion contains: 5% Squalene 5% tocopherol 2.0% tween 80; the particle size is 180 nm.

Preparation of Oil in Water Emulsion (2 Fold Concentrate)

Tween 80 was dissolved in phosphate buffered saline (PBS) to give a 2% solution in the PBS. To provide 100 ml two fold concentrate emulsion 5 g of DL alpha tocopherol and 5 ml of squalene were vortexed until mixed thoroughly. 90 ml of PBS/Tween solution was added and mixed thoroughly. The resulting emulsion was then passed through a syringe and finally microfluidised by using an M110S microfluidics machine. The resulting oil droplets have a size of approximately 180 nm.

1.4—Preparation of Adjuvant Systems.

1.4.1 Adjuvant System A: QS21 and 3D-MPL

A mixture of lipid (such as phosphatidylcholine either from egg-yolk or synthetic) and cholesterol and 3 D-MPL in organic solvent, was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was added to give a final cholesterol concentration of 5 to 50 mg/ml.

The liposomes have a defined size of 100 nm and are referred to as SUV (for small unilamelar vesicles). The liposomes by themselves are stable over time and have no fusogenic capacity. Sterile bulk of SUV was added to PBS to reach a final concentration of 10, 20 or 100 μg/ml of 3D-MPL. PBS composition was Na2HPO4: 9 mM; KH2PO4: 48 mM; NaCl: 100 mM pH 6.1. QS21 in aqueous solution was added to the SUV. This mixture is referred as DQMPLin. Stx-OVA was then added. Between each addition of component, the intermediate product was stirred for 5 minutes. The pH was checked and adjusted if necessary to 6.1+/−0.1 with NaOH or HCl.

In the experiments described in section 3.1 below, StxB-OVA was at a concentration of 4, 10, 20 or 100 μg/ml and 3D-MPL and QS21 were at a concentration of 10 μg/ml. In these cases, the injection volume of 50 μl corresponded to 0.2-5 μg of STxB-OVA and 0.5 μg of 3D-MPL and QS21. The results for an injection of 0.2 μg of STxB-OVA are shown in FIGS. 1-10. Experiments were also carried out where an injection volume of 50 μl corresponded to 0.5, 1 and 5 μg of STxB-OVA. These experiments gave comparable results to those shown in FIGS. 1 to 10.

In other experiments, StxB-OVA was at a concentration of 20 or 40 μg/ml and 3D-MPL and QS21 were at a concentration of 20 or 100 μg/ml.

In these experiments, the injection volume of 25 μl corresponded to 0.5 μg of STXB-OVA and 0.5 μg of 3D-MPL and QS21 (shown in FIGS. 12A and 12B) or 1 μg STxB-OVA and 2.5 μg each 3D-MPL and QS21 (shown in FIGS. 11 and 20)

1.4.2 Adjuvant System B: QS21

1.4.2.1: Adjuvant System B1

The adjuvant was prepared according to the methods used for Adjuvant system A but omitting the 3 D-MPL.

StxB-OVA and QS21 were adjusted at a concentration of 10 or 20 μg/ml.

Injection volumes of 25 or 50 μl corresponded to 0.5 μg of StxB-OVA and 0.5 μg of QS21 (as shown in FIGS. 12A, 12B and 17)

1.4.2.2: Adjuvant System B2

QS21 was diluted at a concentration of 100 μg/ml in PBS pH 6.8 before addition of StxB-OVA to reach a final antigen concentration of 40 μg/ml.

An injection volume of 25 μl corresponded to 1 μg of StxB-OVA and 2.5 μg of QS21 (as shown in FIG. 16)

1.4.3 Adjuvant System C: 3D-MPL

1.4.3.1: Adjuvant System C1

Sterile bulk of 3D-MPL was diluted at 100 or 200 μg/ml in a sucrose solution at a final concentration of 9.25%. StxB-OVA was added to reach an antigen concentration of 20 or 40 μg/ml.

Injection volume of 25 μl corresponded to 1 μg of StxB-OVA and 5 μg of 3D-MPL (seen in FIG. 16) or 0.5 μg of StxB-OVA and 2.5 μg of 3D-MPL (results not shown, but comparable).

1.4.3.2: Adjuvant System C2

The adjuvant was prepared according to the methods used for Adjuvant system A but omitting the QS21.

StxB-OVA and MPL were adjusted to a concentration of 10 μg/ml.

An injection volume of 50 μl corresponded to 0.5 μg of StxB-OVA and 0.5 μg of MPL.

1.4.4 Adjuvant System D: 3D-MPL and QS21 in an Oil in Water Emulsion

Sterile bulk emulsion prepared as in example 1.3 was added to PBS to reach a final concentration of 250 or 500 μl of emulsion per ml (v/v). 3 D-MPL was then added to reach a final concentration of 50 or 100 μg/ml. QS21 was then added to reach a final concentration of 50 or 100 μg per ml. Between each addition of component, the intermediate product was stirred for 5 minutes. StxB-OVA was then added to reach a final concentration of 10 or 40 μg/ml. Fifteen minutes later, the pH was checked and adjusted if necessary to 6.8+/−0.1 with NaOH or HCl.

Injection volume of 25 or 50 μl corresponded to 0.5 or 1 μg of STxB-Ova, 2.5 μg of 3 D-MPL and QS21, 12.5 μl or 25 μl of emulsion. An experiment using a 50 μl injection volume is shown in FIG. 11. The experiment using a 25 μl injection volume gave comparable results.

1.4.5 Adjuvant System E: High Dose 3D-MPL and QS21 in an Oil in Water Emulsion.

Sterile bulk emulsion prepared as in example 1.3 was added to PBS to reach a final concentration of 500 μl of emulsion per ml (v/v). 200 μg of 3D-MPL and 200 μg QS21 were added. Between each addition of component, the intermediate product was stirred for 5 minutes. StxB-OVA was then added to reach a final concentration of 40 μg/ml. Fifteen minutes later, the pH was checked and adjusted if necessary to 6.8+/−0.1 with NaOH or HCl.

Injection volume of 25 μl corresponded to 1 μg of STxB-Ova, 5 μg of both immunostimulants and 12.5 μl emulsion.

1.4.6 Adjuvant System F: 3D-MPL and QS21 in an Low Oil in Water Emulsion.

Oil in water emulsion was as in example 1.3 with cholesterol being added to the organic phase to reach a final composition of 1% squalene, 1% tocopherol, 0.4% tween 80, and 0.05% Cholesterol. After formation of the emulsion, 3 D-MPL was then added to reach a final concentration of 100 μg/ml. QS21 was then added to reach a final concentration of 100 μg per ml. Between each addition of component, the intermediate product was stirred for 5 minutes. StxB-OVA was then added to reach a final concentration of 40 μg/ml. Fifteen minutes later, the pH was checked and adjusted if necessary to 6.8+/−0.1 with NaOH or HCl. Injection volume of 25 μl corresponded to 1 μg of STxB-Ova, 2.5 μg of 3 D-MPL and QS21, 2.5 μl emulsion.

1.4.7 Adjuvant System G: CpG2006

Sterile bulk CpG was added to PBS or NaCl 150 mM solution to reach a final concentration of 100 or 200 μg/ml.

StxB-OVA was then added to reach a final concentration of 10 or 20 μg/ml.

The CpG used was a 24-mers with the following sequence 5′-TCG TCG TTT TGT CGT TTT GTC GTT-3′ (Seq ID No.4). Between each addition of component, the intermediate product was stirred for 5 minutes. The pH was checked and adjusted if necessary to 6.1+/−0.1 with NaOH or HCl.

Injection volume of 50 μl corresponded to 0.5 μg of STxB-Ova and 5 μg of CpG (FIGS. 12A, 12B and 21). Experiments were done with injection volumes of 25 μl (corresponding to 05 μg of STxB-Ova and 5 μg of CpG). Results are not shown but were comparable.

1.4.8 Adjuvant System H: QS21, 3D-MPL and CpG2006

Sterile bulk CpG was added to PBS solution to reach a final concentration of 100 μg/ml. PBS composition was Na₂HPO₄: 9 mM; KH2PO4: 48 mM; NaCl: 100 mM pH 6.1. StxB-OVA was then added to reach a final concentration of 20 μg/ml. Finally, QS21 and 3 D-MPL were added as a premix of sterile bulk SUV containing 3 D-MPL and QS21 referred as DQMPLin to reach final 3D-MPL and QS21 concentrations of 10 μg/ml.

The CpG used was a 24-mers with the following sequence 5′-TCG TCG TTT TGT CGT TTT GTC GTT-3′ (Seq ID No.4). Between each addition of component, the intermediate product was stirred for 5 minutes. The pH was checked and adjusted if necessary to 6.1+/−0.1 with NaOH or HCl.

Injection volume of 50 μl corresponded to 1 μg of STxB-Ova, 0.5 μg of 3 D-MPL and QS21 and 5 μg of CpG. This formulation was then diluted in a solution of 3D-MPL/QS21 and CpG (at a concentration of 10, 10 and 100 μg/ml respectively) to obtain doses of 0.2, 0.04 and 0.008 μg of StxB-OVA. (these formulations used for experiments shown in FIGS. 1 to 10 and 13)

In the experiment shown in FIGS. 12 A and 12B, CpG was at a concentration of 100 μg/ml, 3D-MPL and QS21 at a concentration of 10 μg/ml and StxB-OVA at a concentration of 10 μg/ml.

Injection volume of 50 μl corresponded to 0.5 μg of StxB-OVA, 0.5 μg of 3D-MPL and QS21 and 5 μg of CpG.

In one further experiment, CpG was at a concentration of 1000 μg/ml, 3D-MPL and QS21 at a concentration of 100 μg/ml and StxB-OVA at a concentration of 40 μg/ml. Injection volume of 25 μl corresponded to 1 μg of StxB-OVA, 2.5 μg of 3D-MPL and QS21 and 25 μg of CpG. Results from this experiment are not shown, but are comparable with the results seen with other concentrations of components.

1.4.9 Adjuvant System I: QS21 and CpG2006

Sterile bulk CpG was added to PBS or NaCl 150 mM solution to reach a final concentration of 100 or 200 μg/ml. PBS composition was PO4 10 mM, NaCl 150 mM pH 7.4 or Na2HPO4: 9 mM; KH2PO4: 48 mM; NaCl: 100 mM pH 6.1. StxB-OVA was then added to reach a final concentration of 10 or 20 μg/ml. Finally, QS21 was added as a premix of sterile bulk SUV and QS21 (referred as DQ, prepared as in example 1.3.14) to reach final QS21 concentration of 10 or 20 μ/ml.

The CpG used was a 24-mers with the following sequence 5′-TCG TCG TTT TGT CGT TTT GTC GTT-3′ (Seq ID No.4). Between each addition of component, the intermediate product was stirred for 5 minutes. The pH was checked and adjusted if necessary to 6.1 or 7.4+/−0.1 with NaOH or HCl.

Injection volumes of 50 μl corresponded to 0.5 μg of STxB-Ova, 0.5 μg of QS21 and 5 μg of CpG (FIGS. 12 A and 12B)

Experiments were also done with injection volumes of 25 μl (corresponding 0.5 μg of STxB-Ova, 0.5 μg of QS21 and 5 μg of CpG). Results are not shown but were comparable.

1.4.10 Adjuvant System J: Incomplete Freunds Adjuvant (IFA)

IFA was obtained from CALBIOCHEM. IFA was emulsified with a volume of antigen using vortex during one minute.

STxB-ova was diluted at 40 μg/ml concentration in PBS pH 6.8 or 7.4 and mixed with 500 μl/ml of IFA either used as such or after a 20-fold dilution in PBS.

Injection volume of 25 μl corresponded to 1 μg of STxB-ova and 12.5 or 0.625 μl of IFA (shown in FIG. 14).

In other experiments, StxB-OVA was diluted at 10 μg/ml in PBS pH 6.8 or 7.4 and mixed with 500 or 250 μl/ml of IFA. Injection volume of 50 μl corresponded to 0.5 μg of StxB-OVA and 12.5 or 25 μl of IFA. These experiments gave comparable results to those shown in FIG. 14.

1.4.11 Adjuvant System K: Oil in Water Emulsion

1.4.11.1 Adjuvant System K1

Sterile bulk emulsion was prepared as in example 1.3 except that 3D-MPL and QS21 were omitted.

Injection volume of 25 μl corresponded to 1 μg of StxB-OVA and 12.5 μl of emulsion. Results are shown as adjuvant system K in FIG. 16.

1.4.11.2 Adjuvant System K2

Sterile bulk emulsion was prepared as in Adjuvant system F except that 3D-MPL and QS21 were omitted.

Injection volume of 25 μl corresponded to 1 μg of StxB-OVA and 2.5 μl of emulsion containing Cholesterol.

Results are not shown, but were comparable to those seen with adjuvant system K1.

1.4.12 Adjuvant System L: Poly I:C

Poly I:C (polyinosinic-polycytidylic acid) is a commercial synthetic mimetic of viral RNA from Amersham. In some experiments, StxB-OVA was diluted in NaCl 150 mM to reach a final concentration of 20 μg/ml. Sterile bulk Poly I:C was then added to reach a final concentration of 20 μg/ml.

Between each addition of component, the intermediate product was stirred for 5 minutes.

Injection volume of 25 μl corresponded to 0.5 μg of STxB-Ova and 0.5 μg of Polyl:C (shown in FIGS. 15 and 21)

In other experiments, StxB-OVA was at a concentration of 10 μg/ml and Poly I:C at a concentration of 20 or 100 μg/ml.

Injection volume of 50 μl corresponded to 0.5 μg StxB-OVA and 1 or 5 μg of Poly I:C. These experiments gave comparable results to those shown in FIGS. 15 and 21.

1.4.13 Adjuvant System M: CpG5456

StxB-OVA was diluted in NaCl 150 mM to reach a final concentration of 20 μg/ml. Sterile bulk CpG was then added to reach a final concentration of 200 μg/ml.

The CpG used was a 22-mers with the sequence 5′-TCG ACG ITT TCG GCG CGC GCC G-3′ (CpG 5456). Between each addition of component, the intermediate product was stirred for 5 minutes.

Injection volume of 25 μl corresponded to 0.5 μg of STxB-Ova and 5 μg of CpG.

1.4.14 Adjuvant System N: QS21 and Poly I:C

A mixture of lipid (such as phosphatidylcholine either from egg-yolk or synthetic) and cholesterol in organic solvent, was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was then added to give a final cholesterol concentration of 5 to 50 mg/ml.

The liposomes have a defined size of 100 nm and are referred to as SUV (for small unilamelar vesicles). The liposomes by themselves are stable over time and have no fusogenic capacity.

Sterile bulk of SUV was added to PBS to reach a final concentration of 100 μg/ml of MPL. QS21 in aqueous solution was added to the SUV to reach a final QS21 concentration of 100 μg/ml. This mixture of liposome and QS21 is referred as DQ.

Sterile bulk Poly I:C (Amersham, as before) was diluted in NaCl 150 mM to reach a final concentration of 20 μg/ml before addition of DQ to reach a final concentration of 20 μg/ml in QS21. StxB-OVA was then added to reach a final concentration of 20 μg/ml. Between each addition of component, the intermediate product was stirred for 5 minutes.

Injection volume of 25 μl corresponded to 0.5 μg of STxB-Ova, 0.5 μg of QS21 and 0.5 μg of Polyl:C.

1.4.15 Adjuvant System O: CpG2006 and Oil in Water Emulsion

Oil in water emulsion was prepared as in example 1.3.

Sterile bulk emulsion was added to PBS to reach a final concentration of 500 μl of emulsion per ml (v/v). CpG was then added to reach a final concentration of 200 μg/ml. Between each addition of component, the intermediate product was stirred for 5 minutes. StxB-OVA was then added to reach a final concentration of 20 μg/ml. Fifteen minutes later, the pH was checked and adjusted if necessary to 6.8+/−0.1 with NaOH or HCl.

The CpG used was a 24-mers with the following sequence 5′-TCG TCG TTT TGT CGT ITT GTC GTT-3′ (Seq ID No.4).

Injection volume of 25 μl corresponded to 0.5 μg of STxB-Ova, 5 μg of CpG and 12.5 μl of emulsion.

1.4.16 Adjuvant System P: CpG2006 and Oil in Water Emulsion

An oil-in-water emulsion was prepared following the recipe published in the instruction booklet contained in Chiron Behring FluAd vaccine.

A citrate buffer was prepared by mixing 36.67 mg of citric acid with 627.4 mg of Na citrate 2H2O in 200 ml H2O. Separately, 3.9 g of squalene and 470 mg of Span 85 were mixed under magnetic stirring.

470 mg of Tween 80, was mixed with the citrate buffer. The resulting mixture was added to the squalene/Span 85 mixture and mixed “vigorously” with magnetic stirring. The final volume was 100 ml.

The mixture was then put in the M110S microfluidiser (from Microfluidics) to reduce the size of the oil droplets. A z average mean of 145 nm was obtained with a polydispersity of 0.06. This size was obtained on the Zetasizer 3000HS (from Malvern) using the following technical conditions:
laser wavelength: 532 nm (Zeta3000HS).
laser power: 50 mW (Zeta3000HS).
scattered light detected at 90° (Zeta3000HS).
temperature: 25° C.,
duration: automatic determination by the soft,
number: 3 consecutive measurements,
z-average diameter: by cumulants analysis

Sterile bulk of the resulting emulsion was added to PBS to reach a final concentration of 500 μl of emulsion per ml (v/v). CpG was then added to reach a final concentration of 200 μg/ml. Between each addition of component, the intermediate product was stirred for 5 minutes. StxB-OVA was then added to reach a final concentration of 20 μg/ml. Fifteen minutes later, the pH was checked and adjusted if necessary to 6.8+/−0.1 with NaOH or HCl.

The CpG used was a 24-mers with the following sequence 5′-TCG TCG TTT TGT CGT TTT GTC GTT-3′ (Seq ID No.4)

Injection volume of 25 μl corresponded to 0.5 μg of STxB-Ova, 5 μg of CpG and 12.5 μl emulsion.

1.4.17 Adjuvant System Q: CPG2006 and IFA Water in Oil Emulsion

IFA, obtained from CALBIOCHEM, was added to PBS to reach a final concentration of 500 μl of emulsion per ml (v/v). CpG was then added to reach a final concentration of 200 μg/ml. Between each addition of component, the intermediate product was stirred for 5 minutes. StxB-OVA was then added to reach a final concentration of 20 μg/ml. Fifteen minutes later, the pH was checked and adjusted if necessary to 7.4+/−0.1 with NaOH or HCl.

The CpG used was a 24-mers with the following sequence 5′-TCG TCG TTT TGT CGT TTT GTC GTT-3′ (Seq ID No.4)

Injection volume of 25 μl corresponded to 0.5 μg of STxB-Ova and 5 μg of CpG, 12.5 μl emulsion.

1.4.18 Adjuvant System R: CPG2006 and Al(OH)3

Al(OH)₃ from Brentag was diluted at final concentration of 1 mg/ml (Al+++) in water for injection. StxB-OVA was adsorbed on Al+++ at a concentration of 20 μg/ml during 30 minutes. CpG was added to reach a concentration of 200 μg/ml and incubated for 30 minutes before addition of NaCl to reach a final concentration of 150 mM. All incubations were performed at room temperature under orbital shacking

The CpG used was a 24-mers with the following sequence 5′-TCG TCG TTT TGT CGT TTT GTC GTT-3′ (Seq ID No.4)

Injection volume of 25 μl corresponded to 0.5 μg of STxB-Ova, 5 μg of CpG and 25 μg of Al+++.

1.4.19 Adjuvant System S: CPG2006 and AlPO4

AlPO4 from Brentag was diluted at final concentration of 1 mg/ml (Al+++) in water for injection. STxB-OVA was adsorbed on Al+++ at a concentration of 20 μg/ml during 30 minutes. CpG was added to reach a concentration of 200 μg/ml and incubated for 30 minutes before addition of NaCl to reach a final concentration of 150 mM. All incubations were performed at room temperature under orbital shacking The CpG used was a 24-mers with the following sequence 5′-TCG TCG TTT TGT CGT TTT GTC GTT-3′ (Seq ID No.4)

Injection volume of 25 μl corresponded to 0.5 μg of STxB-Ova, 5 μg of CpG and 25 μg of Al+++.

1.4.20 Adjuvant System T: 3D-MPL and Al(OH)3

Al(OH)3 from Brentag was diluted at a final concentration of 1 mg/ml (Al+++) in water for injection. StxB-OVA was adsorbed on Al+++ at a concentration of 40 or 20 μg/ml during a 30-minute period. 3D-MPL was added to reach a concentration of 100 μg/ml and incubated for 30 minutes before addition of NaCl to reach a final concentration of 150 mM. All incubations were performed at room temperature under orbital shaking

Injection volume of 25 μl corresponded to 1 or 0.5 μg of STxB-Ova, 2.5 μg of 3D-MPL and 25 μg of Al+++. Results for 1 μg of STxB-Ova are shown in FIG. 16. Experiments where 0.5 μg STxB-Ova were injected are not shown, but gave comparable results to that shown in FIG. 16.

1.4.21 Adjuvant System U: TLR2-Ligand

The TLR2 ligand used was a synthetic Pam3CysSerLys4, a bacterial lipopeptide purchased from Microcollections which is known to be TLR2 specific. StxB-OVA was diluted in NaCl 150 mM or in PBS pH 7.4 to reach a final concentration of 10 or 20 μg μg/ml. Sterile bulk Pam3CysSerLys4 was then added to reach a final concentration of 40, 100 and 200 μg/ml. Between each addition of component, the intermediate product was stirred for 5 minutes.

Injection volume of 50 μl corresponded to 0.5 μg of STxB-Ova and 5 or 10 μg of Pam3CysSerLys4. (Results for 5 μg shown in FIG. 21, see section 3.2.9 for discussion of results with other doses of TLR2)

In other experiments, injection volume of 25 μl corresponded to 0.5 μg of StxB-OVA and 1 μg of Pam3CysSerLys4.

1.4.22 Adjuvant System V: TLR7/8 Ligand.

The TLR 7/8 ligand used was an imiquimod derivative known as resiquimod or R-848 (Cayla). R-848 is a low molecular weight compound of the imidazoquinoline family that have potent anti-viral and anti-tumor properties in animal models. The activity of imiquimod is mediated predominantly through the induction of cytokines including IFN-a and IL-12. R-848 is a more potent analogue of imiquimod (Akira, S. and Hemmi, H.; IMMUNOLOGY LETTER, 85, (2003), 85-95).

STxB-OVA was diluted in PBS pH 7.4 to reach a final concentration of 10 or 20 μg/ml. Sterile bulk R-848 was then added to reach a final concentration of 20 and 100 μg/ml. Between each addition of component, the intermediate product was stirred for 5 minutes.

Injection volume of 50 μl corresponded to 0.5 μg of STxB-Ova and 1 or 5 μg of R-848. In other experiment, injection volume of 25 μl corresponded to 0.5 μg of STxB-OVA and 0.5 μg of R-848.

1.4.22 Adjuvant System W: AlPO4.

1.4.22.1 Adjuvant System W1

AlPO4 from Brentag was diluted at final concentration of 0.5 mg/ml (Al+++) in water for injection. STxB-OVA was adsorbed on Al+++ at a concentration of 10 μg/ml during 30 minutes before addition of NaCl to reach a final salt concentration of 150 mM. All incubations were performed at room temperature under orbital shacking Injection volume of 50 μl corresponded to 0.5 μg of STxB-Ova and 25 μg of Al+++.

1.4.22.2 Adjuvant System W2

AlPO4 from Brentag was diluted in PBS pH 7.4 at final concentration of 0.5 mg/ml (Al+++). STxB-OVA was adsorbed on Al+++ at a concentration of 10 μg/ml during 30 minutes. All incubations were performed at room temperature under orbital shacking Injection volume of 50 μl corresponded to 0.5 μg of STxB-Ova, and 25 μg of Al+++. Examination by SDS-PAGE as set out in XXXXX indicated that about 70% of the antigen was not adsorbed onto the AlPPO4

1.5 Determination of Level of Adsorbed Antigen in an Antigen/Metal Salt Complex

The formulation of interest is centrifuged for 6 min at 6500 g. A sample of the resulting supernatant is denatured for 5 minutes at 95° C., and loaded onto an SDS-PAGE gel in reducing sample buffer. A sample of the antigen without adjuvant is also loaded. The gel is then run at 200V, 200 mA for 1 hour. The gel is then silverstained according to the Daichi method. Levels of free antigen in the formulation are determined by comparing the sample from the adjuvanted formulation with the antigen without adjuvant. Other techniques that are well known in the art, such as Western blotting, may also be used.

### Example 2

### Vaccination of C57/B6 Mice with Vaccines of the Invention

Various formulations as described above were used to vaccinate 6-8 week old C57BL/B6 female mice (10/group). The mice received two injections spaced 14 days apart and were bled during weeks 1, 2, 3 and 4 (for actual bleed days see specific examples) The mice were vaccinated intramuscularly (injection into the left gastrocnemien muscle of a final volume of 50 μl). The ovalbumin recombinant adenovirus was injected at a dose of 1 to 5×10⁸ VP.

Ex-vivo PBLs stimulation were performed in complete medium which is RPMI 1640 (Biowitaker) supplemented with 5% FCS (Harlan, Holland), 1 μg/ml of each anti-mouse antibodies CD49d and CD28 (BD, Biosciences), 2 mM L-glutamine, 1 mM sodium pyruvate, 10 μg/ml streptamycin sulfate, 10 units/ml penicillin G sodium (Gibco), 10 μg/ml streptamycin 50 μM B-ME mercaptoethanol and 100× diluted non-essential amino-acids, all these additives are from Gibco Life technologies. Peptide stimulations were always performed at 37° C., 5% CO2.

2.1 Immunological Assays:

2.1.1 Detection of Antigen-Specific T Cells

Isolation of PBLs and tetramer staining. Tetramer is available only for the ovalbumine antigen model (ova), the siinfekl-tetramer is commercially available (Immunomics Coulter). Blood was taken from retro-orbital vein (50 μl per mouse, 10 mice per group) and directly diluted in RPMI+heparin (LEO) medium. PBLs were isolated through a lymphoprep gradient (CEDERLANE). Cells were then washed, counted and finally 3×10⁵ cells were re-suspended in 50 μl FACS buffer (PBS, FCS1%, 0.002% NaN3) containing CD16/CD32 antibody (BD Biosciences) at 1/50 final concentration (f.c.). After 10 min., 50 μl of the tetramer mix was added to cell suspension. The tetramer mix contains 1 μl of siinfekl-H2 Kb tetramer-PE from Immunomics Coulter and anti-CD8a-PercP ( 1/100 f.c.) antibodies were added in the test. The cells were then left for 10 minutes at 37° C. before being washed once and analysed using a FACS Calibur™ with CELLQuest™ software, 3000 events within the gate of living CD8 are required per test.

2.1.2 Intracellular Cytokine Staining (ICS).

ICS was performed on blood samples taken as described in paragraph 2.1.1. This technology is applied for both antigen-models: ova and HBS.

10⁶ PBLs were re-suspended in complete medium supplemented with either a pool of 15-mer HBS peptides (54 peptides covering the whole HBS sequence used at f.c. of 1 μg/ml of each peptide) when needed or a pool of 17 15-mer Ova peptides (11 MHC classI-restricted peptides and 6 MHC classII-restricted peptides) present at a concentration of each 1 μg/ml. After 2 hours, 1 μg/ml Brefeldin-A (BD, Biosciences) was added for 16 hours and cells were collected after a total of 18 hours. Cells were washed once and then stained with anti-mouse antibodies all purchased at BD, Biosciences; all further steps were performed on ice. The cells were first incubated for 10 min. in 50 μl of CD16/32 solution ( 1/50 f.c., FACS buffer). 50 μl of T cell surface marker mix was added ( 1/100 CD8a perCp, 1/100 CD4 APCcy7) and the cells were incubated for 20 min. before being washed. Cells were fixed & permeabilized in 200 μl of perm/fix solution (BD, Biosciences), washed once in perm/wash buffer (BD, Biosciences) before being stained at 4° C. with anti IFNg-APC anti IL2-FITC and anti TNFa-PE either for 2 hours or overnight. Data were analysed using a FACS Calibur™ with CELLQuest™ software, 15000 events within the gate of living CD8 are required per test.

2.1.3 Cell Mediated Cytotoxic Activity Detected In Vivo (CMC In Vivo).

This technology is applied for both antigen-models: ova and HBS.

To assess antigen-specific cytotoxic activity, immunized and control mice were injected with a mixture of targets. This mixture consists of 2 or 3 differentially CFSE-labeled syngeneic splenocyte and lymphnode populations, loaded or not (as mentioned on graphs). Target are loaded with adequate antigen: 1 nM siinfekl peptide or HBS peptide pool (pool of 54 peptides at a f.c. 1 μg/ml each peptide). For the differential labeling, carboxyfluorescein succinimidyl ester (CFSE; Molecular Probes—Palmoski et al.; 2002, J. Immunol. 168, 4391-4398) was used at a concentration of 0.05 μM, 0.5 or 5 μM. The different types of targets (2 or 3) were pooled at 1/1 ratio and re-suspended at a concentration of 10⁸ targets/ml. 200 μl of target mix were injected per mouse into the tail vein 15 days after 1^{st} injection. Cytotoxic activity was assessed by FACS^{R} analysis in blood (jugular vein) taken from sacrificed animal 18H after target injection. The mean percentage lysis of each antigen specific-loaded target cells was calculated relative to antigen-negative controls with the following formula:

$$lysis \% = 100 - ( \frac{corrected target ( + )}{control target ( - )} \times 100 )$$

$$C orrected target += target + x \frac{( preinj . - )}{( pre \text{-} inj . + )}$$

Pre-injected target cells=mix of ad hoc peptide-pulsed targets (pre-inj.+) and non-pulsed (pre-inj.−) targets acquired by FACS before injection in vivo.

Corrected target (+)=number of ad hoc peptide-pulsed targets acquired by FACS after injection in vivo, corrected in order to take into account the number of pre-inj+ cells in the pre-injected mix (see above).

2.1.4 Ag Specific Antibody Titer (Individual Analysis of Total IgG): ELISA.

This technology is applied for both antigen-models: ova and HBS.

Serological analysis was assessed 15 days. Mice (10 per group) were bled by retro-orbital puncture. Anti-HBS and Anti-ova total IgG were measured by ELISA. 96 well-plates (NUNC, Immunosorbant plates) were coated with antigen overnight at 4° C. (either 50 μl per well of HBS solution (HBS 10 μg/ml, PBS) or 50 μl per well of ova solution (ova 10 μg/ml, PBS). The plates were then washed in wash buffer (PBS/0.1% Tween 20 (Merck)) and saturated with 100 μl of saturation buffer (PBS/0.1% Tween 20/1% BSA/10% FCS) for 1 hour at 37° C. After 3 further washes in the wash buffer, 100 μl of diluted mouse serum was added and incubated for 90 minutes at 37° C. After another three washes, the plates were incubated for another hour at 37° C. with biotinylated anti-mouse total IgG diluted 1000 times in saturation buffer. After saturation 96w plates were washed again as described above. A solution of streptavidin peroxydase (Amersham) diluted 1000 times in saturation buffer was added, 50 μl per well. The last wash was a 5 steps wash in wash buffer. Finally, 50 μl of TMB (3,3′,5,5′-tetramethylbenzidine in an acidic buffer-concentration of H₂O₂ is 0.01%-BIORAD) per well was added and the plates were kept in the dark at room temperature for 10 minutes

To stop the reaction, 50 μl of H₂SO₄ 0.4N was added per well. The absorbance was read at a wavelength of 450/630 nm by an Elisa plate reader from BIORAD. Results were calculated using the softmax-pro software.

2.1.5 B Cell Elispot

Spleen and bone marrow cells were collected at 78 days after 2^{nd} injection and cultured at 37° C. for five days in complete medium supplemented with 3 μg/ml of CpG 2006 and 50 U/ml of rhIL-2 to cause memory B cells to differentiate into antibody-secreting plasma cells. After five days, 96-well filter plates were incubated with ethanol 70% for 10 minutes, washed, and coated with either ovalbumin (50 μg/ml) or an a goat anti-mouse Ig antiserum. They were then saturated with complete medium. Cells were harvested, washed and dispatched on the plates at 2×10⁵ cells/well for one hour at 37° C. The plates were then stored overnight at 4° C. The day after, the cells were discarded by washing the plates with PBS Tween 20 0.1%. The wells were then incubated at 37° C. for one hour with an anti-IgG biotynilated antibody diluted in 1/500 PBS, washed and incubated for one hour with extravidin-horseradish peroxidase (4 μg/ml). After a washing step, the spots were revealed by a 10 minute incubation with a solution of amino-ethyl-carbazol (AEC) and H₂O₂ and fixed by washing the plates with tap water. Each cell that has secreted IgG or Ova-specific IgG appears as a red spot. The results are expressed as frequency of ova-specific IgG spots per 100 total IgG spots.

### 3. Results

The results described below show that the efficiency of the STxB system at inducing CD8 responses was dramatically improved by combining it with various adjuvant systems or some of their components.

3.1 Data with Adjuvant Systems A & H

3.1.1 Evaluation of the Primary Response with AS A and AS H

The results obtained show that low dose (0.2 μg) immunization with STxB-ova in the absence of adjuvant does not induce a strong CD8 T cell immune response that can be detected ex-vivo. By contrast, a strong immune response is observed when STXB-OVA is combined with either adjuvant system A or H. Furthermore a clear advantage is demonstrated over the adjuvanted protein.

STxB-ova adjuvanted with adjuvant system A or H is potent at inducing a strong and persistent primary response. It induces high frequency of antigen-specific CD8 T cells (FIG. 1—injections included 0.2 μg of STxB-OVA, 0.5 μg of 3D-MPL and QS21, and 5 μg CPG for AS H. Methods carried out as described in 2.1.1 above, mice were bled at 7 days after 1^{st} injection). In addition, FIG. 2 (injections included 0.2 μg of STxB-OVA, 0.5 μg of 3D-MPL and QS21, and 5 μg CPG for AS H. Methods carried out as described in 2.1.1 above, mice were bled at 14 days after 1^{st} injection) shows that this siinfekl-specific CD8 response still increases between day 7 and day 14 after injection. This is not observed upon vaccination with the adjuvanted protein, but is rather characteristic of the primary response induced by a live vector such as adenovirus. The primed CD8 T cells are readily differentiated effector T cells, which produce IFNγ whether the stimulation is performed with the immunodominant peptide or a pool of ova peptides (respectively shown in FIGS. 3 and 4, injections included 0.2 μg of STxB-OVA, 0.5 μg of 3D-MPL and QS21, and 5 μg CPG for AS H. Methods carried out as described in 2.1.2 above, mice were bled at 14 days after 1^{st} injection). The higher frequency of responder CD8 T cells observed upon restimulation with the peptide pool indicates that the primary CD8 T cell repertoire is not limited to the class I immunodominant epitope. In addition, high cytotoxic activity can be detected in vivo only when STxB-ova is adjuvanted (FIG. 5—injections included 0.2 μg of STxB-OVA, 0.5 μg of 3D-MPL and QS21, and 5 μg CPG for AS H. Methods carried out as described in 2.1.3 above at 18 hours following target injection).

Finally the primary response induced by AS H adjuvanted STxB-ova is strongly persistent, as illustrated in FIG. 6B (injections included 0.2 μg of STxB-OVA, 0.5 μg of 3D-MPL and QS21, and 5 μg CPG. methods carried out as described in 2.1.1 above, mice were bled at different time points).

3.1.2 Evaluation of the Secondary Response with AS A and AS H

Combining the STxB toxin delivery system with potent adjuvants also improves amplitude and persistence of the secondary immune response. This is best exemplified by evaluating the response 47 days after the boost. Importantly, the high CD8 response induced by the adjuvanted STxB-OVA is of similar intensity and persistence as that induced by a recombinant adenovirus prime/adjuvanted protein boost strategy (FIG. 6A—injections included 0.2 μg of STxB-OVA, 0.5 μg of 3D-MPL and QS21, and 5 μg CPG for AS H. Methods carried out as described in 2.1.1 above, mice bled 47 days following 2^{nd} injection). Regarding effector T-cell population, cytokine-producing T cells are still detected in both CD4 and CD8 T cell compartments (FIGS. 7 and 8—injections included 0.2 μg of STxB-OVA, 0.5 μg of 3D-MPL and QS21, and 5 μg CPG for AS H. Methods carried out as described in 2.1.2 above, mice were bled 47 days following 2^{nd} injection, PBLs were stimulated with a pool of ova peptides). Moreover, at this late time point, a cytotoxic activity can still be detected in vivo 4 hours (data not shown), and 24 hours (FIG. 9—injections included 0.2 μg of STxB-OVA, 0.5 μg of 3D-MPL and QS21, and 5 μg CPG for AS H. Methods carried out as described in 2.1.3 above) after target injection.

The humoral response has been investigated 15 days and 40 days after boost (FIG. 10a—injections included 0.2 μg of STxB-OVA, 0.5 μg of 3D-MPL and QS21, and 5 μg CPG for AS H. Methods carried out as described in 2.1.4 above, results shown through the geomean calculation for each group of 10 mice). In the absence of adjuvant, STxB-ova alone is unable to induce any B cell response. By contrast, equivalent antibody titers are detected whether the adjuvanted protein is coupled to STxB or not at both time points tested.

In FIG. 10B (injections included 0.2 μg of STxB-OVA, 0.5 μg of 3D-MPL and QS21, and 5 μg CPG. methods carried out as described in 2.1.5 above) the anti-ova memory B cell frequency is shown 78 days post injection. Although the antibody titers detected 15 and 40 days after two injections are equivalent, the quality of the memory B cell response is different as a higher frequency of memory B cells is detected when STxB-ova is adjuvanted as compared to adjuvanted protein. STxB-ova alone is unable to induce memory B cell on its own.

Interestingly, when priming and boost are given 42 days instead of 14 days apart (FIG. 20—injection included 0.5 μg of STXB-OVA and 0.5 μg of 3D-MPL and QS21, methods carried out as in 2.1.4 above), humoral response induced by STxB-OVA AS A is higher than OVA AS A, again suggesting that when combined with adjuvantation, vectorisation may induce a higher frequency of B cell memory cells.

3.1.3 Evaluation of the Immune Response Induced by Low Doses of StxB-Ova Combined with the AS H Adjuvant System

FIG. 13 (injections included 0.008, 0.04, 0.2 or 1 μg of STxB-OVA, 0.5 μg of 3D-MPL and QS21, and 5 μg CPG. Methods carried out as described in 2.1.1 above, mice bled 14 days after 1^{st} injection) shows that a siinfekl-specific CD8 population can still be detected 14 days after a single injection of doses as low as 8 ng of STxB-ova, corresponding to 4 ng of antigen, formulated in AS H. These results show that the combined use of adjuvant and STxB system could allow a significant reduction of antigen dose without decreasing the induced T cell response.

3.2 Evaluation of the Immune Response Induced by StxB-Ova Combined with Other Adjuvant Systems.

We next wanted to find out whether adjuvant systems other than AS A or AS H could also synergise with the STxB vectorization system.

3.2.1 Evaluation of the Immune Response Following Vaccination with as A, F, D or E STxB Ova Vaccines.

The evaluation of the primary response clearly indicates that an adjuvanted STxB-ova induces a high frequency of antigen specific TCD8 (FIG. 11—methods carried out as described in 2.1.1 above, mice bled at 13 days after 1^{st} injection), whatever the adjuvant system tested. Remarkably, this is seen even with AS D and AS E for which no detectable CD8 response can usually be detected after a single immunization with adjuvanted protein. The adjuvanted STxB-ova strongly primes CD8 T cells which are readily differentiated into cytokine-secreting effector T cells (data not shown).

3.2.2 Evaluation of the Immune Response Induced by StxB-Ova Combined with Individual Components of Adjuvant Systems (3 D-MPL-AS C2, QS21-AS B, CpG2006-AS G)

We next evaluated the different component of the previous adjuvant systems in vivo. FIG. 12A (methods carried out as described in 2.1.1 above, mice bled at 15 days after 1^{st} injection) shows that the a siinfekl-specific CD8 population can be detected if STxB-ova is adjuvanted with a single immunostimmulant such as QS21 or a TLR9-ligand such as CpG and to a lesser extent with a TLR-4 ligand such as 3 D-MPL (AS C2), this latter immunostimulant been even more efficient when used as higher dose (AS C1) as in FIG. 16. As above, these primed CD8 T cells are readily differentiated cytokine-secreting effector cells (data not shown). The secondary CD8 responses induced by each adjuvant component alone are equivalent, but higher responses are observed when STxB-ova is adjuvanted with a combination of QS21 and at least one TLR ligand (FIG. 12B—methods carried out as described in 2.1.1 above, mice bled at 6 days after 2^{nd} injection).

3.2.3 Evaluation of the Immune Response Induced by StxB-OVA Combined with Adjuvant J or Adjuvant K

In contrast to previous published observations, increase of CD8 response is also observed when STxB-OVA is combined with emulsion such as IFA. Formulation with IFA, a water in oil emulsion, increases CD8 responses in a dose dependent manner. Increased frequency of siinfekl-specific CD8 T cells (FIG. 14A) corresponds to improved CD8 effector functions such as cytokine production (FIG. 14B) and cytotoxic activity (FIG. 14C). Similar results are obtained when STxB-ova is combined with an oil in water emulsion

3.2.4 Evaluation of the Immune Response Induced by StxB Ova Combined with Adjuvant System C1, B, K, F or T

We next evaluated AS T and the different components of adjuvant system F. FIG. 16 shows that when combined to STxB-OVA, each component is able to increase the siinfekl-specific CD8 T response. However, the highest response is observed when the components are associated in the formulation.

3.2.5 Evaluation of the Immune Response Induced by StxB Ova Combined with Adjuvant L, G or M.

FIG. 15 shows that combination of STX-B-OVA with TLR ligands such as poly I:C (TLR3) or CpG sequences (TLR9) representative of categories B and C significantly increases the amplitude of the siinfekl specific CD8 T response.

3.2.6 Evaluation of the Immune Response Induced by StxB Ova Combined with Adjuvant System B, N or I

FIG. 17 shows that CD8 response induced by STxB-OVA is clearly improved when adjuvanted with either QS21 alone or QS21 combined with a TLR3 ligand (poly I:C) or a TLR9 ligand (CpG).

3.2.7 Evaluation of the Immune Response Induced by StxB Ova Combined with Adjuvant System G, O, P or Q

FIG. 18 shows that the CD8 response induced by STxB-OVA is clearly improved when adjuvanted with either CpG alone or CpG combined with IFA or with different oil-in-water emulsions.

3.2.8 Evaluation of the Immune Response Induced by StxB Ova Combined with Adjuvant System G, R or S

FIG. 19 shows that the CD8 response induced by STX-B-OVA is clearly improved when adjuvanted with either CpG alone or CpG combined with Al(OH)₃ or AlPO4.

3.2.9 Evaluation of the Immune Response Induced by StxB Ova Combined with Adjuvant System G, L, U or V

FIG. 21 shows that, in addition to TLR9 and 3 ligands, combination of STX-B-OVA with TLR2 and TLR7/8 ligands also significantly increases the amplitude of the siinfekl specific CD8 T response. TLR2 ligand was tested at a range of doses from 0.2 to 10 μg. No increase was seen at doses below 5 μg. Interestingly, a reduced response was seen when the dose was increased to 10 μg. This could be explained by the ability of TLR2 ligand to induce regulatory molecules such as IL-10.

3.2.10 Evaluation of the Immune Response Induced by StxB Ova Combined with Adjuvant System W1 or W2.

FIG. 22 shows that the combination of STxB-Ova with AS W1 (which contains aluminium phosphate in a formulation in which the antigen is adsorbed onto the aluminium salt) gives little improvement in the immune response over that seen with unadjuvanted STxB-ova peptide. However, when the composition is formulated such that some of the antigen (in this case about 70%) is not adsorbed onto the aluminium salt, for example by performing the adsorption with aluminium salt dissolved in phosphate buffered saline as is seen in AS W2, then an improvement in immune response is seen over that given by STxB-Ova without adjuvant.

3.2.11 Evaluation of the Immune Response Induced by a Composition Comprising Ova Conjugated to StxB, HBs as Free Antigen, and Adjuvant System A

FIGS. 23-31 evaluate the immune response to two antigens—ova conjugated to Stx, and yeast-produced and purified recombinant Hepatitis B surface protein (HBs) included as free antigen in the same composition. The composition was adjuvanted with adjuvant system A. The whole adaptive immune response was examined, antibodies were measured against both antigens (FIG. 31), tetramer read outs were taken (FIG. 23) and cytotoxic activity measured (FIG. 30). In addition, CD4 and CD8 responses were measured at 7 and 14 days post 1^{st} injection and 7 days post second injection (FIGS. 24-29). Responses are shown as total cytokine (IFNg/TNFa/IL2) producing T cells.

The tetramer read outs show that siinfekl specific responses can be seen when HBs is present as free antigen, therefore confirming that the presence of free antigen does not interfere with the immune response to the conjugated antigen.

Cytokine responses were seen at all timepoints to both antigens, although the primary response (CD4 and CD8) and ova specific CD4 are very low. The CD8 ova specific response induced by the STxB-conjugated ova is high in all vaccine that included that conjugate. As anticipated, Ova specific CD4 response was lower than the CD8 response. HBs and Ova specific T cell responses were both detectable in the seconday response measured at 7 days post 2^{nd} injection. A positive impact of the HBs antigen can be seen on the ova-specific T-Cell response induced by the adjuvanted vector.

Both antigens show cytotoxic activity (measured in vivo on day 26 post 2^{nd} injection, 16 hours after targets injection) and generate humoral responses measured 15 days post 2^{nd} injection. This shows that the presence of free or conjugated antigen does not impede with the immune response seen to the other antigen.

What is claimed is:

1. A vaccine composition comprising the B subunit of Shiga toxin or an immunologically functional equivalent thereof which is able to bind the Gb3 receptor, complexed with at least one first antigen, and further comprising at least one second antigen and an adjuvant.

2. A vaccine composition as claimed in claim 1 wherein the immunologically functional equivalent of the B subunit of Shiga toxin has at least 50% amino acid sequence identity to the B subunit of Shiga toxin.

3. A vaccine composition as claimed in claim 2, wherein the vector is the B subunit of Shiga toxin or a functional fragment thereof.

4. A vaccine composition as claimed in claim 2 wherein the vector is the B subunit of Verotoxin-1 or a functional fragment thereof

5. A vaccine composition as claimed in claim 1 wherein the adjuvant is selected from the group of metal salts, oil in water emulsions, Toll like receptor ligands, saponins or combinations thereof.

6. A vaccine composition as claimed in claim 5 wherein the adjuvant is a Toll like receptor ligand.

7. A vaccine composition as claimed in claim 6 wherein the adjuvant is a Toll like receptor agonist.

8. A vaccine composition as claimed in claim 1, wherein the antigen and B sub unit are covalently attached.

9. A vaccine composition as claimed in claim 8 wherein the antigen is attached to the toxin via a cysteine residue.

10. A vaccine composition as claimed in claim 1 wherein the adjuvant is selected from the group: metallic salts, a saponin, lipid A or derivative thereof, an alkyl glucosamininde phosphate, an immunostimulatory oligonucleotide or combinations thereof.

11. A vaccine composition as claimed in claim 10 wherein the saponin is presented in the form of a liposome, Iscom, or an oil in water emulsion.

12. A vaccine composition as claimed in claim 10 wherein the saponin is QS21.

13. A vaccine composition as claimed in claim 10, wherein the Lipid A derivative is selected from Monophosphoryl lipid A, 3 deacylated Monophosphoryl lipid A, an alkyl glucosaminide phosphate, OM 174, OM 197, OM 294.

14. A vaccine composition as claimed in claim 1 wherein the adjuvant is a combination of at least one representative from two of the following groups,
i) a saponin,
ii) a Toll-like receptor 4 ligand, and
iii) a Toll-Like receptor 9 ligand.

15. A vaccine composition as claimed in claim 14 wherein the saponin is QS21 and the toll like receptor 4 ligand is 3 deacylated monophosphoryl lipid A and the toll like receptor 9 ligand is a CpG containing immunostimulatory oligonucleotide.

16. A vaccine composition as claimed in claim 1 wherein at least one first antigen and at least one second antigen are the same antigen.

17. A vaccine composition as claimed in claim 16 wherein the antigen is selected from the group of antigens that provide immunity against the group of diseases selected from, intracellular pathogens or proliferative diseases.

18. A vaccine composition as claimed in claim 1 wherein at least one first antigen and at least one second antigen are different.

19. A vaccine composition as claimed in claim 18 wherein the first antigen is NS3 from HCV.

20. A vaccine composition as claimed in claim 19 wherein the second antigen is E1 from HCV.

21. A vaccine composition comprising the B subunit of Shiga toxin or an immunologically functional equivalent thereof with at least one first antigen, at least one second antigen and an adjuvant for use in medicine.

22. (canceled)

23. (canceled)

24. A method of treating or preventing disease comprising administering to a patient suffering from or susceptible to disease a vaccine composition according to claim 1.

25. A method for raising an antigen specific CD 8 immune response comprising the administration to a patient of a vaccine according to claim 1.

26. A process for the production of a vaccine according to claim 1 wherein an antigen in combination with the B subunit of shiga toxin or immunologically functional equivalent thereof is admixed with a further antigen and an adjuvant.


SEQUENCE LISTING (6 sequences)

SEQ ID NO: 1 - DNA, 20 bp, Artificial Sequence
  Immunostimulatory oligonucleotide CpG 1826
tccatgacgttcctgacgtt

SEQ ID NO: 2 - DNA, 18 bp, Artificial Sequence
  Immunostimulatory oligonucleotide CpG 1758
tctcccagcgtgcgccat

SEQ ID NO: 3 - DNA, 30 bp, Artificial Sequence
  Immunostimulatory oligonucleotide
accgatgacgtcgccggtgacggcaccacg

SEQ ID NO: 4 - DNA, 24 bp, Artificial Sequence
  Immunostimulatory oligonucleotide CpG 2006
tcgtcgttttgtcgttttgtcgtt

SEQ ID NO: 5 - DNA, 20 bp, Artificial Sequence
  Immunostimulatory oligonucleotide CpG 1668
tccatgacgttcctgatgct

SEQ ID NO: 6 - DNA, 22 bp, Artificial Sequence
  Immunostimulatory oligonucleotide CpG 5456
tcgacgttttcggcgcgcgccg